SEQUENCE LISTING (11 sequences)

SEQ ID NO: 1 - protein, 284 aa, Farfantepenaeusaztecus
MDAIKKKMQAMKLEKDNAMDRADTLEQQNKEANNRAEKSEEEVHNLQKRMQQLENDLDQVQESLLKANIQLVEKDKALSNAEGEVAALNRRIQLLEEDLERSEERLNTATTKLAEASQAADESERMRKVLENRSLSDEERMDALENQLKEARFLAEEADRKYDEVARKLAMVEADLERAEERAETGESKIVELEEELRVVGNNLKSLEVSEEKANQREEAYKEQIKTLTNKLKAAEARAEFAERSVQKLQKEVDRLEDELVNEKEKYKSITDELDQTFSELSGY

SEQ ID NO: 2 - DNA, 29 bp, artificial
  Primer
caccatggacgccatcaagaagaagatgc

SEQ ID NO: 3 - DNA, 21 bp, artificial
  Primer
agagagggccttgtccttgtc

SEQ ID NO: 4 - DNA, 27 bp, artificial
  Primer
caccatgaacatccagcttgtggagaa

SEQ ID NO: 5 - DNA, 21 bp, artificial
  Primer
gttctcgagcaccttgcgcat

SEQ ID NO: 6 - DNA, 23 bp, artificial
  Primer
caccatgaaggtgctcgagaacc

SEQ ID NO: 7 - DNA, 20 bp, artificial
  Primer
ctcctcagcacgctcaaggt

SEQ ID NO: 8 - DNA, 20 bp, artificial
  Primer
caccatggaccttgagcgtg

SEQ ID NO: 9 - DNA, 21 bp, artificial
  Primer
ctcagccgccttcagcttgtt

SEQ ID NO: 10 - DNA, 31 bp, artificial
  Primer
caccatgcagattaagacacttaccaacaag

SEQ ID NO: 11 - DNA, 26 bp, artificial
  Primer
gtagccagacagttcgctgaaagtct

(19) United States
(12) Patent Application Publication
(10) Pub. No.: US 2017/0296652 A1
(43) Pub. Date: Oct. 19, 2017

(54) METHODS AND COMPOSITIONS INVOLVING RECOMBINANT HYPOALLERGENS

(71) Applicant: STC.UNM, Albuquerque, NM (US)

(72) Inventors: Bridget S. Wilson, Albuquerque, NM (US); Diane Lidke, Albuquerque, NM (US); Lydia Tapia, Albuquerque, NM (US); Mark Schulyer, Albuquerque, NM (US); Avanika Mahajan, Albuquerque, NM (US)

(21) Appl. No.: 15/509,605

(22) PCT Filed: Sep. 17, 2015
(86) PCT No.: PCT/US2015/050700

§ 371 Date: Mar. 8, 2017

Related U.S. Application Data
(60) Provisional application No. 62/051,616, filed on Sep. 17, 2014.

(51) Int. Cl. A61K 39/35; C07K 14/435; C07K 14/415

(52) U.S. Cl. CPC A61K 39/35; C07K 14/415; C07K 14/43509

(57) ABSTRACT

This disclosure describes recombinant hypoallergens and methods of treating allergy that involve administering a recombinant hypoallergen to a subject. Generally, the recombinant hypoallergen includes at least one amino acid modification compared to a corresponding wildtype allergen. As a result, the recombinant hypoallergen binds to IgE that specifically binds to the allergen, but induces release of histamine from basophils to a degree less than the wildtype allergen.

### CROSS-REFERENCE TO RELATED APPLICATION

This application claims priority to U.S. Provisional Patent Application Ser. No. 62/051,616, filed Sep. 17, 2014, which is incorporated herein by reference.

### GOVERNMENT FUNDING

This invention was made with government support under P50GM085273 awarded by the National Institutes of Health. The government has certain rights in the invention.

### SEQUENCE LISTING

This application contains a Sequence Listing electronically submitted to the United States Patent and Trademark Office via EPS-Web as an ASCII text file entitled “2015-09-17-SequenceListingST25.txt” having a size of 5 KB and created on Sep. 15, 2015. Due to the electronic filing of the Sequence Listing, the electronically submitted Sequence Listing serves as both the paper copy required by 37 CFR §1.821(c) and the CRF required by §1.821(e). The information contained in the Sequence Listing is incorporated by reference herein.

### SUMMARY

This disclosure describes, in one aspect, a recombinant hypoallergen. Generally, the recombinant hypoallergen includes at least one amino acid modification compared to a corresponding wildtype allergen. As a result, the recombinant hypoallergen binds to IgE that specifically binds to the allergen, but induces release of histamine from basophils to a degree less than the wildtype allergen.

In some embodiments, the recombinant hypoallergen may be derived from Pen 1 a of Farfantepenaeus aztecus. In some of these embodiments, the hypoallergen can include amino acids 1-79 of SEQ ID NO:1; amino acids 68-127 of SEQ ID NO:1; amino acids 121-181 of SEQ ID NO:1; amino acids 172-236 of SEQ ID NO:1; or amino acids 224-284 of SEQ ID NO:1.

In some embodiments, the recombinant hypoallergen may be derived from Ara h 2 from Arachi hypogaea.

In some embodiments, the recombinant hypoallergen may be derived from Jun a 1 from Juniperus ashei.

In some embodiments, the recombinant hypoallergen may be derived from Fel d 1 of Felis domesticus.

In some embodiments, the recombinant hypoallergen may be derived from Par j 1 of Parietaria judaica.

In another aspect, this disclosure describes a pharmaceutical composition that includes any embodiment of recombinant hypoallergen summarized above and a pharmaceutically acceptable carrier.

In another aspect, this disclosure describes a method of treating allergy in a subject to an allergen. Generally, the method includes administering to the subject an amount of any embodiment of recombinant hypoallergen summarized above in an amount effective to ameliorate at least one symptom or clinical sign of allergy to the allergen.

In yet another aspect, this disclosure describes the use of any embodiment of recombinant hypoallergen summarized above in the manufacture of a pharmaceutical composition for the treatment of allergy to a corresponding wildtype allergen.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Recombinant Expression of Pen a 1, Pen a 1-N and Pen a 1-C. The proteins were expressed with His tags in E. Coli using IPTG induction and purified using Ni-NTA column. Samples were run on SDS PAGE and analyzed with anti-HisTag immunoblotting.

FIG. 2. Structural Analysis of Pen a 1. CD spectroscopy was performed for samples in PBS. Recombinant Pen a 1 showed a typical spectrum for α-helixes with minima at 208 and 222 that was found to be comparable to that obtained for natural Pen a 1.

FIG. 3. Pen a 1-N and Pen a 1-C induce minimal cross-linking of FcεRI and little or no cell activation. RBL-2H3 cells expressing the human alpha subunit of FcεRI were primed with serum of atopic individual (or Human IgE, negative control) and stimulated with recombinant Pen a 1, Pen a 1-N and Pen a 1-C (concentrations shown in legend).

FIG. 4. Pen a 1-N and Pen a 1-C bind IgE from serum of atopic patient. Immunoblot analysis of IgE binding to Pen a 1 and its fragments with serum from control (not allergic) and atopic (shrimp allergic) individual. Bound IgE was detected using HRP conjugated anti-IgE. Arrows point to monomers/dimers of recombinant proteins.

FIG. 5. Illustration of mechanism of immune response to an allergenic antigen (Ag).

FIG. 6. (A) Illustration of QD tracking of IgE-FcεRI complexes. (B) Immobilization of FcεRI is indicated by left-shift in this CPA plot. Higher valency antigens (DNP₁₂-BSA or DNP₂₅-BSA) induce slowdown while low valency ligands (DNP₂-BSA, DNP₄-BSA) show little change in diffusion vs resting conditions. All ligands induce secretion.

FIG. 7. (A) Structure of the trivalent ligand, DF3. (B) DF3 bound to 1 Fab in an IgE-FcεRI complex. (C) Illustration of predicted center-to-center distances of FcεRI α in a cyclic aggregate. (D) Bell-shaped degranulation response to DF3.

FIG. 8. (A) Immunolabeling of membrane sheet ripped from mast cell activated with DF3. (B) Proximity ligation assay for SHIP recruitment to FcεRI (spots) after DF3-mediated crosslinking.

FIG. 9. (A) Pen a 1 (shrimp tropomyosin, homology model); (B) Ara h 2 from peanut, Arachi hypogaea; (C) Jun a 1 from the mountain cedar, Juniperus ashei. Structural analysis by Tapia based on PDB, with IgE epitopes indicated by contrasting colors.

FIG. 10. (A) Use of rule-based model to predict aggregation kinetics for DF3. (B) All atomistic model of 7 IgE-FcεRI bound to 5 DF3 ligands. (C) Graph theory applied to the same complex. (D) Example of graph predictions applied to electron micrograph TEM image of anti-FcεRI gold-labeled membrane after DF3 stimulation.

FIG. 11. Classes of crosslinked IgE-FcεRI aggregates for bivalent ligand and bivalent receptor.

FIG. 12. One of many possible aggregate configurations for IgE-FcεRI with Pen a 1 (shrimp tropomyosin, helical rod).

FIG. 13. (A) Secretion from humanized RBL-49 cells after priming with serum from 3 shrimp-allergic subjects and challenge with tropomyosin. B) Secretory responses to DF3 in RBL-2H3 cells are modified after overnight priming with different IgE.

FIG. 14. Examples of assays for (A) calcium responses; (B) phosphorylation kinetics; (C) Fluorescent in situ hybridization for cytokine mRNA; (D) Receptor internalization. (E) Real time observation of FcεRI aggregation, after priming with two colors of IgE-QD (QD585, QD655) Boxes show segmentation of trajectories into monomer (right), preformed dimer (middle), and trimer (left). Estimated diffusion constants are shown for each segment.

FIG. 15. Basophil isolation.

FIG. 16. General scheme for flow-based multicolor assay for allergen-specific IgE on the surface of live basophils.

FIG. 17. Schematic illustrating systematic mutation of IgE binding epitopes to alter valency and allergenicity of recombinant allergens.

FIG. 18. Threshold and dose-dependency of Pen a 1-mediated response. Pen a 1-specific IgE (IgETM) was purified from the plasma of a shrimp allergic individual by affinity purification. hRBL-2H3 (humanized Rat Basophilic Leukemia) cells express the human FcεRI a subunit. The endogenous rat α was knocked out by CRISPR technology. Cells were primed with purified IgETM. FcεRI-IgE complexes were cross-linked with a range of anti-IgE or Pen a1 concentrations and degranulation responses measured. Error bars represent Standard Deviation. Results are representative of three independent experiments.

FIG. 19. Design and recombinant expression of Pen a1 and fragments. Amino acid sequence of Pen a 1 and the five fragments FR1, FR2, FR3, FR4, and FR5. Sequence does not include N-terminal His tag.

FIG. 20. Characterization of Pen a 1 and fragments. (A) SDS-PAGE. Size-exclusion chromatography purified proteins were separated and detected by coomassie staining. (B) Anti-His western blotting. Size-exclusion chromatography purified proteins were separated and detected by probing with anti-His antibodies. (C) CD spectroscopy in PBS. Intact Pen a 1 showed a typical spectrum for a helix with minima at 208 and maxima at 222 nm. Fragments displayed varying levels of a helix. (D) Immunoblot analysis of IgE binding to rPen a 1 and its fragments with serum from control (not allergic) and atopic (shrimp allergic) individual. Bound IgE was detected using HRP conjugated anti-IgE.

FIG. 21. Pen a 1 fragments elicit minimal degranulation from mast cells and inhibit responses to intact Pen a 1. hRBLrko cells were primed with serum of atopic individual and stimulated with recombinant Pen a 1 and fragments (concentrations shown in legend). Humanized RBL-2H3 cells were incubated with serum from shrimp allergic individual for two hours before addition of allergens. (A) Cells stimulated with recombinant Pen a 1, isolated fragments or fragments pooled together (concentrations shown in legend). (B) Addition of fragment mix in specified concentrations to cells pre-incubated with 1 μg/ml Pen a 1 for five minutes. Degranulation response is based on the percent of total β-hexosaminidase released from cells after stimulation for 30 minutes.

### DETAILED DESCRIPTION OF ILLUSTRATIVE EMBODIMENTS

This disclosure describes methods for creating hypoallergens as a therapeutic strategy for shrimp allergy and compositions that include the hypoallergens.

Allergic mediators are released after an allergen binds to allergen-specific IgE complexed with FcεRI on mast cells or basophils. Circulating levels of IgE are generally quite low in unaffected or mildly atopic individuals, since high affinity binding supports stable IgE-FcεRI complexes. Although the field acknowledges that the mast cell or basophil surface is “decorated” with a repertoire of pre-bound IgE, there has been very little attempt to quantify the levels of allergen-specific IgE and dose of antigen needed to reach a signaling threshold in human cells. There also is a lack of information about the critical structural features of allergens that determine mast cell and basophil responsiveness.

This disclosure describes recombinant forms of allergens that activate an allergic response to a lesser degree than the native form of the allergen. The recombinant forms of the allergens can be used in therapeutic methods for treating an allergic immune response directed against the native form of the allergen. Using novel computational approaches, one can evaluate the influence of allergen valency, epitope affinity, and/or spacing on antigen-IgE-FcεRI aggregation. One also can analyze human FcεRI signaling in response to allergen-mediated crosslinking using humanized cell lines and primary human basophils—and link this back to aggregation kinetics through mathematical modeling.

Allergy and its associated diseases are common health problems in the developed world. These conditions include for example, asthma, food allergies, allergic rhinitis, atopic dermatitis, and antibiotic reactions, some of which can be life-threatening. The US Center for Disease Control estimates that >50 million Americans are affected. This disclosure focuses on, as model allergens, two common food allergens: peanut and shellfish.

The approach described herein focuses on the structural, biophysical, and signaling features of human FcεRI when it is bound to polyvalent antigen. A food allergen, shrimp tropomyosin (Pen a 1) was used as a model allergen to evaluate allergen-specific responses in human FceRIa-transfected cell lines and primary human basophils. The analyses described herein may be applied to other allergens such as, for example, the environmental allergen, Jun a 1 from Juniperus ashei, Fel D1 from cat, and peanut allergens such as, for example, Ara h 2.

Allergen-specific IgE in serum and on the surface of freshly isolated basophils can be quantitated, providing parameters for mathematical models that link response measures (e.g., histamine release and/or cytokine transcription) to receptor engagement at specific doses. Characterization of antigen-IgE-FcεRI aggregate size, receptor orientation and spacing, and signaling output can be obtained using conventional assays and/or computational methods.

The FcεRI tetrameric receptor belongs to the Immunoreceptor Super Family that includes the T cell receptor (TCR), B cell antigen receptor (BCR), activating Fcγ receptors, and a growing list of other leukocyte receptors. A common feature of these receptors is the presence of one or more ITAMs (immunoreceptor tyrosine-based activation motif) that recruit signaling molecules after tyrosine phosphorylation. Due to the role of ITAM-based signaling in the immune system, the structural “dissection” of FcεRI crosslinking for efficient human basophil signaling may have broad impact for the immunology field.

This disclosure describes using imaging, refined quantitative measures, and computational approaches to understand how authentic allergens crosslink and activate the human FcεRI. For example, one can apply high resolution imaging approaches to evaluate the diffusion and redistribution of human FcεRI on the membranes of allergen-challenged human primary cells and connect these events to signal propagation, desensitization, and/or internalization. Rigorous statistical tools and computational modeling methods enable one to link receptor behavior and topology to functional outputs such as, for example, degranulation and/or cytokine production. One novel emphasis is the analysis of allergen structure as it relates to crosslinking FcεRI into aggregates that favor the activation of positive signaling molecules (e.g., Syk, PLCγ, PI3K, LAT, etc.) versus the engagement of negative regulatory players (e.g., SHIP, SHP, CBP/PAG, DOK, Cbl, etc.). The central hypothesis of allergy is that soluble allergens all share a few basic criteria: crosslinking epitopes spaced within narrow distance constraints on the 3D surface, limited flexibility, and epitope affinities in the low nM range. The methods described herein exploit the conformational and aggregation limitations of the FcεRI for signal initiation and propagation. The methods described herein can be used to design new immunotherapy reagents that compete with allergens for available receptors while causing poor or negligible signaling responses.

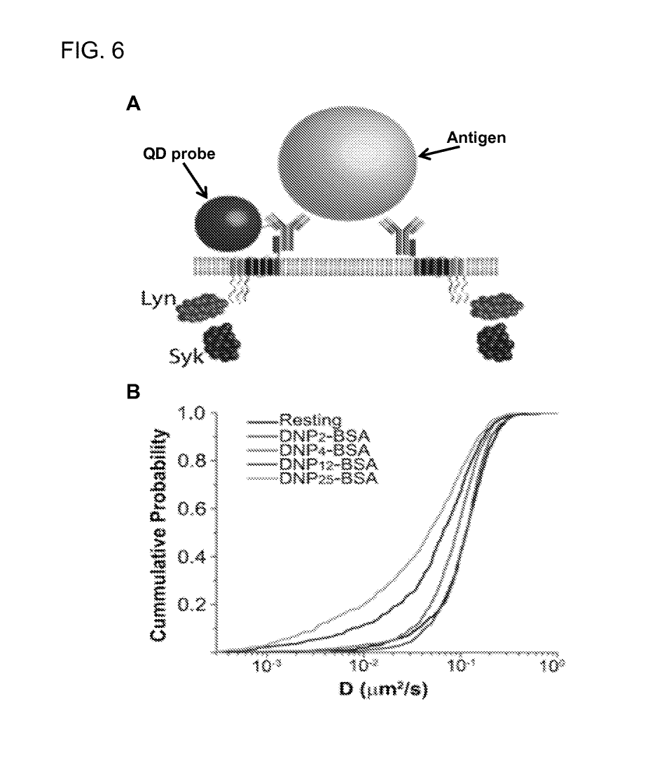

This work used quantum dot (QD)-IgE probes for single particle tracking of FcεRI leading to the observation that small aggregates of crosslinked FcεRI are both signaling competent and mobile (FIG. 6). Since early biophysical studies had linked FcεRI signaling to the immobilization of receptors, this was an unexpected finding. Using single molecule tracking methods, it was also possible to further refine the conditions under which receptors slow down and reach a state where a large fraction of surface FcεRI are essentially immobile. Mechanistically, the immobilization process appears to be complex. It reflects a combination of a) restricted diffusion due to the physical size of the antigen-IgE-FcεRI complex, which further expands as signaling proteins dock on the cytoplasmic tails, and b) the capture of receptors into clathrin-coated pits and other budding endocytic carriers. Aggregate size and immobilization can be controlled by lowering the dose of a highly polyvalent antigen and/or by lowering the valency of haptens on the carrier used to crosslink dinitrophenyl (DNP)-specific IgE on FcεRI. Mast cells often respond relatively poorly to bivalent ligands and do not degranulate in response to soluble monovalent ligands. An exception to the monovalent ligand rule occurs in the context of the mast cell synapse, when thousands of FcεRI migrate to engage ligand on a lipid bilayer.

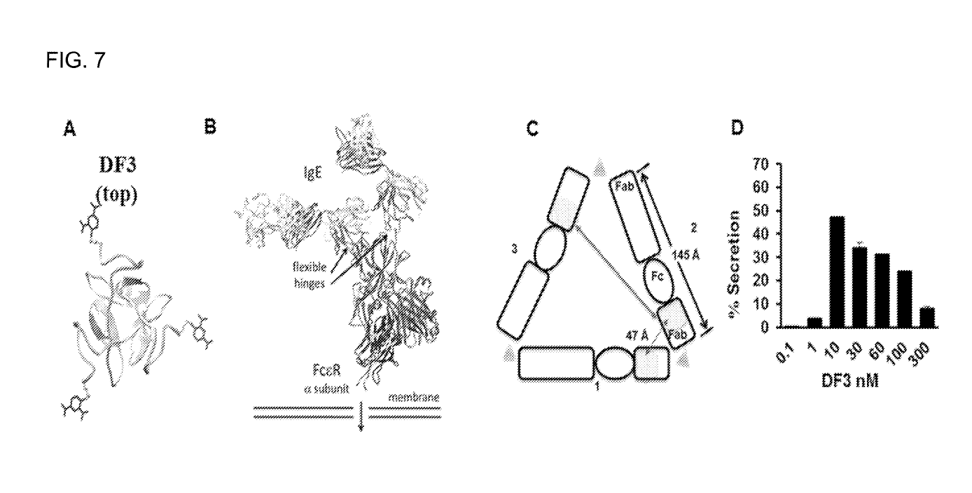

A trivalent DNP ligand (referred to herein as DF3) was designed and synthesized to aid in the study of allergen valency and structure. DF3 is based on the foldon peptide domain of fibritin, which spontaneously folds into a stable trimer (FIG. 7A). High resolution microscopy methods were combined with modeling approaches at two different scales. At the finest computational scale, detailed knowledge about IgE bound to the FcεRI α subunit and the structure of foldon allows one to predict the docking of DF3 onto IgE-FcεRI complexes. Several observations came from the modeling studies. First, since IgE is oriented at an angle when bound to FcεRI, the complex is not symmetrical about its axis (FIG. 7B). Second, each of the three haptens in DF3 can bind a Fab “arm” of IgE and engage up to three IgE-FcεRI simultaneously. Since each IgE is bivalent, another DF3 can be engaged through the free Fab arm. Chains and complex aggregates form as a result. Third, the model predicts significant variability in the predicted center-to-center distances between receptors (using the a subunit as a marker) within complexes. The simplest case is illustrated in FIG. 7C, for a cyclic aggregate (3 IgE-FcεRI: 3 DF3).

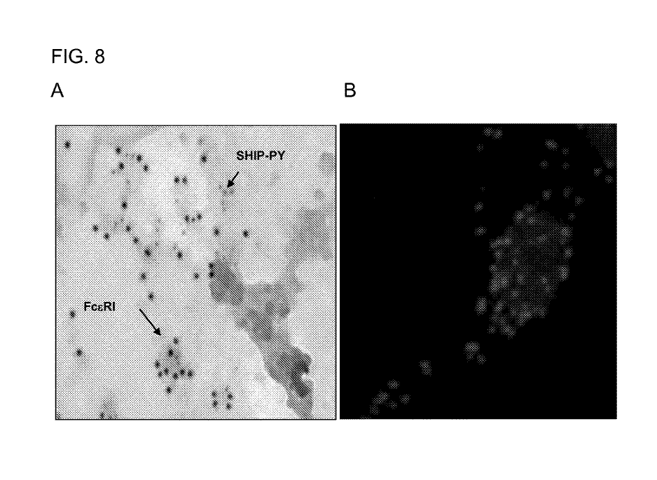

The degranulation response to DF3 is bell-shaped (FIG. 7D). In the past, the progressive loss of responsiveness with increasing polyvalent ligand concentration has been termed “high dose inhibition.” The prevailing explanation for this phenomenon in human basophils has been that excess antigen opposes crosslinking through a prevalence of monovalent interactions. This notion was challenged through modeling and experimentation. Mathematical modeling, suggested that >80% of receptors persist in some form of aggregate at inhibitory doses of DF3 (Mahajan et al., 2014, ACS Chem. Biol. 9(7):1508-1519). Electron microscopy and DUOLINK (Olink Bioscience, Uppsala, Sweden) proximity ligation assays revealed that poor signaling at both ends of the bell-shaped curve is associated with differential recruitment of the inositol phosphatase, SHIP, to the FcεRI signaling patch (FIG. 8). These new observations suggest that inhibitory partners are recruited preferentially under some crosslinking conditions support the view that activating ITAMs can be converted to ITAMi (inhibitory ITAMs).

Databases (e.g., Allergome and SDAP) provide access to information about known allergenic proteins, as well as tools for predicting cross-reactive peptides. Over 2000 allergens important to humans are cataloged so far, with 3D models available for ˜450 antigens and identification of major protein families. One can compare structural features in nine important Pfam families, with the understanding that cross-reactive allergens are often from the same structural family. For example, the profilins of pollen and plant food can sensitize allergic individuals for latex reactions. Similarly, the EF-hand motifs of grass, tree, and/or weed pollens can mediate clinically observed cross-reactivity to a broad group of plant antigens. For example, Pen a 1 and related tropomyosins of crustaceans and molluscs are highly allergenic. Their similarity to dust mite tropomyosins mean that exposure to dust mites can sensitize atopic individuals for reactivity to seafood, even in subjects with little or no pre-exposure to the antigen in food. Peptide mapping of cross-reactive allergens began over two decades ago, leading to classification of IgE-reactive short peptide sequences within allergens as linear epitopes. Conformational epitopes also have been identified, which are defined by greater dependency on protein 3D structure. Moreover, linear and conformational mapping of common IgE-binding epitopes on antigens has been performed for several allergens. In addition, peptide epitope motif databases can be mined and/or 3D structures can be analyzed for discontinuous epitopes.

Nevertheless, and despite these important bioinformatics resources, there is not yet a matching well of information about the impact of allergen structure on crosslinking of IgE-FcεRI and the subsequent triggering of inflammatory mediator release from mast cells and basophils. This disclosure describes this type of investigation, focusing initially on two food allergens and one environmental allergen as model allergens. The model allergens were selected based upon their expected incidence in the pool of local donors, overall importance to human health, and their dissimilar structural features.

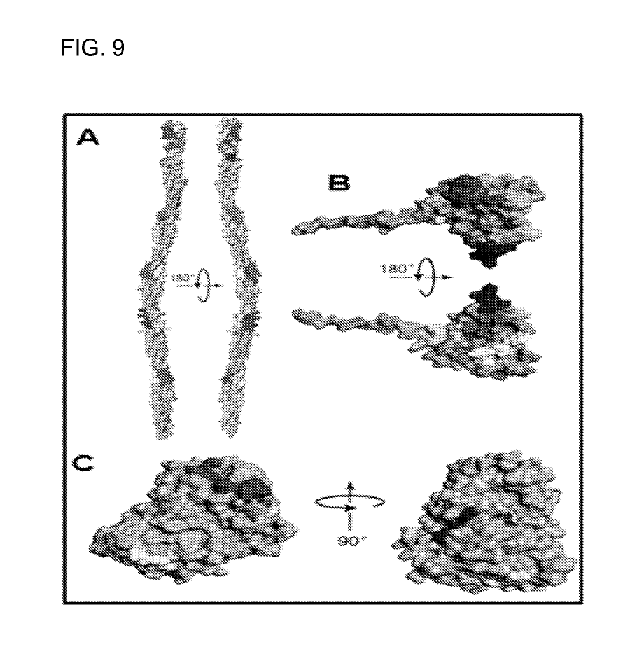

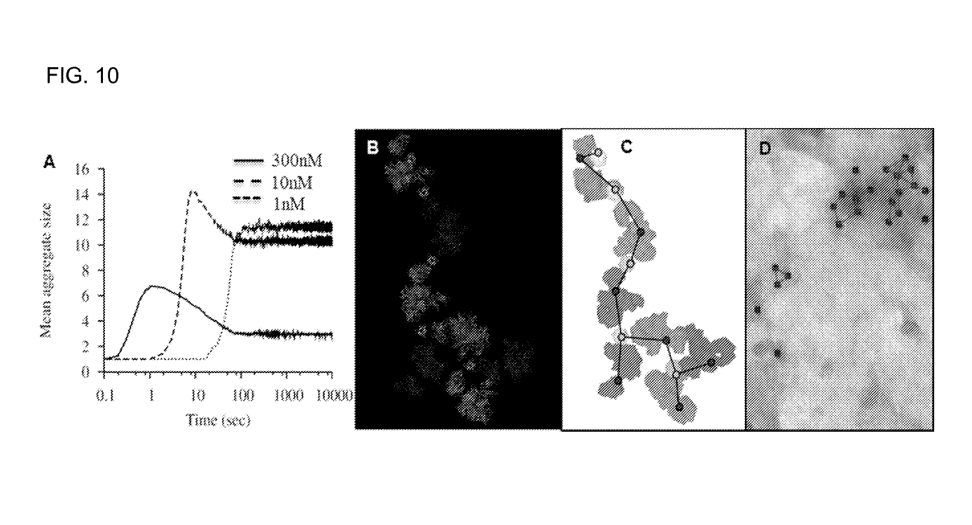

One allergen is Pen a 1 (shrimp tropomyosin; FIG. 9A and FIG. 10). Available atomic-level structural information about the allergen provided estimates of the proximity of individual FcεRI after aggregation by allergen. Using complementary modeling approaches of aggregate formation, the extent to which the spacing, composition, and/or kinetics of allergen-IgE-FcεRI complexes determine activation of mast cells and basophils by antigen was analyzed. The model can be validated in humanized rat basophilic leukemia (RBL) cells that express the human FcεRI a subunit. Finally, the effects of allergen-mediated crosslinking on other parameters such as, for example, signaling, diffusion, and/or internalization were measured.

A second model allergen is the food allergen Ara h 2 from peanut. A third model allergen is Jun a 1 pollen from juniper. A fourth model allergen is Fel d 1 from cats. The identification of the key allergen features for triggering FcεRI can serve as the foundation for developing new hypoallergens for immunotherapy. By definition, hypoallergens stimulate release of Th₂-type cytokines and other inflammatory mediators from mast cells and basophils to a degree less than the native allergen on which the hypoallergen is based. A hypoallergen can retain a sufficient repertoire of B cell and T cell epitopes to increase specific regulatory T cells and induce protective IgG responses.

The degree to which a hypoallergen stimulates an allergic response (e.g., release of histamine, prostaglandins, Th₂-type cytokines, chemokines and/or other inflammatory mediators) can vary. A hypoallergen may stimulate an allergic response to a degree that is lower than—in some cases even absent compared to—the native allergen on which the hypoallergen is based.

One can evaluate allergen aggregating capacity, protein rigidity, and/or relative distance and orientation of FcεRI within aggregates, and then correlate these features with FcεRI signaling. Quantitative measures (Table 1) can include the recruitment and activity of signaling partners, calcium signaling, and downstream responses (e.g., degranulation, leukotriene/prostaglandin production and cytokine mRNA).

TABLE 1
Imaging, biochemical, and functional assays

| Assay | Citation |
|---|---|
| In vitro kinase assays | Wilson et al., 1995, J Biol Chem |
| | 269: 29697-29703 |
| Phosphorylation kinetics | Oliver et al., 1994, J Biol Chem |
| | 269: 29697-29703; Youssef et al., |
| | 2007, J Immunol 178: 4584-4594; |
| | Mahajan et al., 2014 ACS Chem |
| | Biol 9(7): 1508-1519 |
| PI 3-kinase, PLCγ activity | Barker et al., 1998, Mol Biol Cell |
| | 9: 483-496; Barker et al., 1999, J. |
| | Leukoc Biol. 65: 321-329; Smith et |
| | al., 2001, J Biol Chem 276: 17213-17220 |
| Degranulation, arachidonic | Wilson et al., 1989, J Immunol |
| acid release | 143: 259-265 |
| Cytokine measures (ELISA) | Gilmartin et al., 2008, Int Arch |
| | Allergy Immunol 145: 182-192 |
| Gene expression profiling | Youssef et al., 2007, J Immunol |
| | 178: 4584-4594; Hernandez- |
| | Hansen, J Immunol 175: 7880-7888 |
| Proximity ligation, | Mahajan et al., 2014 ACS Chem |
| co-precipitation | Biol 9(7): 1508-1519; Wilson et al., |
| | 1995, J Biol Chem 269: 29697-29703 |
| Calcium ratio imaging, | Smith et al., 2001, J Biol Chem |
| FRET biosensors | 276: 17213-17220 |
| Flow-based measurements of | Cleyrat et al., 2013, Science |
| receptor internalization, | 126: 4913-4925; Barker et al., |
| actin assembly | 1998, Mol Biol Cell 9: 483-496 |
| Receptor diffusion, real | Andrews et al., 2009, Immunity |
| time aggregation protein | 31: 469-479; Carroll-Portillo et al., |
| translocation (TIRF, SPT) | 2010, J Immunology 184: 1328-1338 |

### Structural Modeling of Allergen-IgE-FcεRI α Complex to Predict Receptor Orientation in Aggregates.

FIG. 7, FIG. 9, and FIG. 10 demonstrate the integration of structural information about IgE-FcεRI complexes and their target allergens from multiple computational scales. The first step is an in silico docking of the Fab arm(s) of an IgE-FcεRI complex onto allergen epitopes. Where possible, the model is built upon all atomistic information. However, only a few structures are available for IgE-allergen interactions at this level and none exist for the three test allergens. On the other hand, since the epitopes are mapped on these allergens, it is possible to develop useful, lower-resolution predictions of the aggregates. All available information is incorporated at this stage, including homology modeling and information derived from any prior mutagenesis or biochemical studies.

Two complementary methods allow one to identify aspects of the aggregation process. One approach is an extension of the Goldstein-Perelson model (Goldstein, B. and Perelson, A., 1984, Biophys J. 45:1109-1123). Parameterization is based upon equilibrium binding data of fluorescent ligand. The model accounts for ligand capture from solution (K₁) and receptor crosslinking (K₂), while growth of “super aggregates” is constrained by accounting for diffusional limitations of larger aggregates. FIG. 10A illustrates the kinetics of aggregate formation, as well as changing sizes of aggregates, for three different doses of the trivalent ligand DF3 (from Mahajan et al., 2014 ACS Chem Biol 9(7):1508-1519). The model is adaptable to higher valency ligands, to ligands with a range of different affinities for individual epitopes, and/or to different IgE repertoires. The computational methods are fast and, because they are rule-based, are adaptable to many variables.

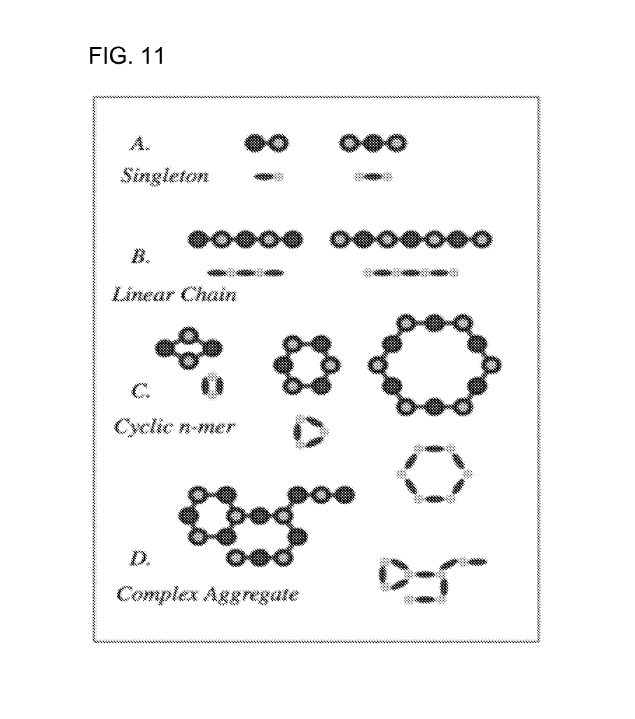

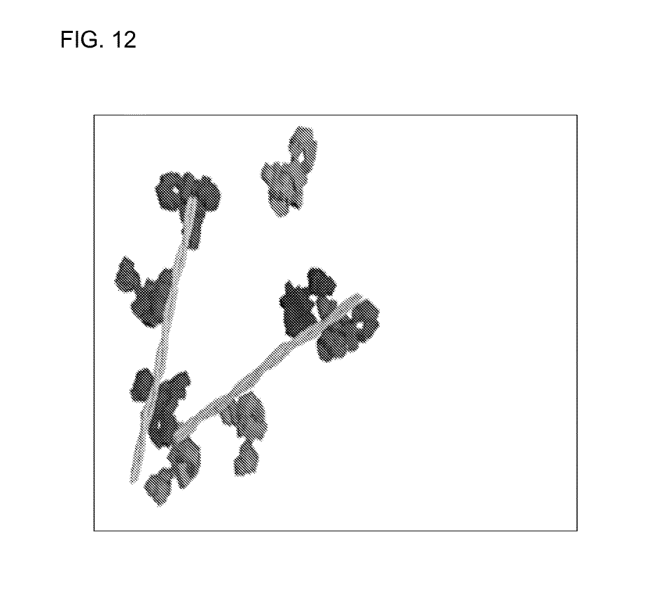

FIG. 10B-D illustrates an agent-based simulation that reveals the full range of aggregate configurations as they form, break apart, and rearrange. FIG. 10B shows an atomistic view of seven IgE-FcεRI complexes bound to five of the trivalent DF3 ligands. FIG. 10C shows how this aggregate is depicted by polygonal reductions of the IgE and ligand molecules. The molecular interactions of these 3D structures can be modeled using Monte Carlo simulations and a graph-based approach. Simulations are initialized with randomly placed distributions for molecules, which diffuse on the XY plane and rotate about the Z axis. At each step, every pair of molecules within a defined binding distance bind with a probability assigned by the association rate. Fab arms dissociate from ligand with probabilities based on the experimentally determined dissociation rate constant. The line in FIG. 10C traces nodes of each molecule in this large complex. The outcome is a graphical map of all possible aggregate configurations between a bivalent IgE and a given multivalent ligand. This compilation can be compared with immunogold-labeled FcεRI on cell membranes after crosslinking with ligand. This is illustrated in FIG. 10D, where dots mark the locations of gold nanoparticles and lines represent possible cyclic and complex aggregate configurations (FIG. 11).

### Quantitative Imaging and Biochemical Assays for Measures of Receptor Aggregation, Diffusion, Signaling and Internalization in Response to Allergen Challenge.

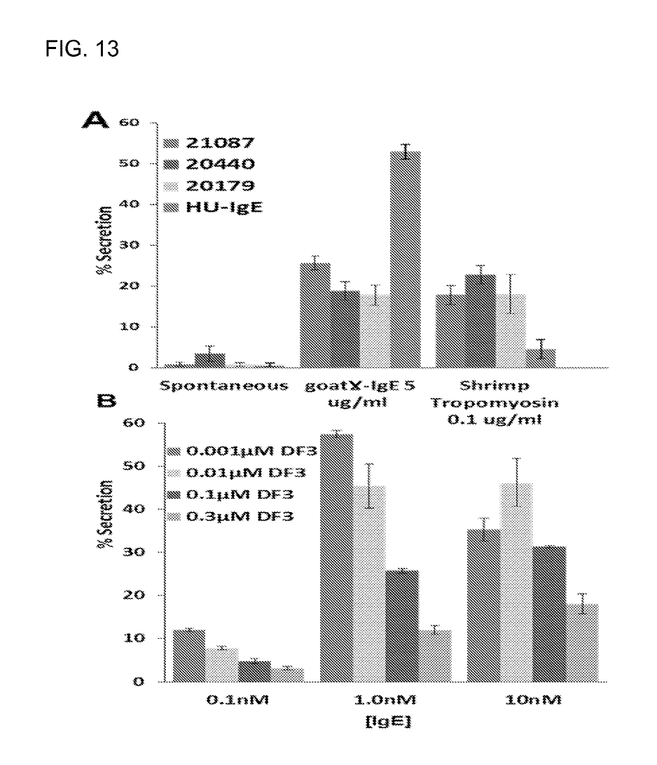

As shown in FIG. 13A, the first test allergen is markedly different in overall structure from artificial ligands previously studied. Shrimp tropomyosin is a dimeric filament, with five major IgE-binding epitope clusters on each of the monomers. FIG. 12 shows preliminary results of Monte Carlo simulations for aggregation of bivalent IgE-FcεRI complexes with decavalent tropomyosin. Aggregates formed by this allergen have the potential to be highly branched and complex. The epitopes are spaced from 6.7-10.3 nm apart (avg. spacing 8 nm), with a ˜32 nm span between the epitopes near each end.

In FIG. 13A provides a model system that one can use to study signaling after crosslinking of FcεRI by tropomyosin. FIG. 13 reports secretory responses in humanized (hRBL) cells that stably express the human α subunit of FcεRI, which forms a heterotetramer with endogenous ITAM-bearing β and γ₍₂₎ subunits. These mast cells were primed with serum from three shrimp-allergic subjects (21087, 20440, and 20179). After washing, the cells were challenged with either anti-IgE (positive control) or tropomyosin and supernatants collected for measuring histamine release. Bars in this plot show results with cells primed with a commercial myeloma-derived human IgE (HU-IgE). They secrete robustly to anti-IgE challenge but not to tropomyosin.

As shown in FIG. 13B, defined concentrations IgE (e.g., affinity purified IgE from serum of a shrimp-allergic subject) can be used to control the number of receptors available for crosslinking and alter the dose response to DF3 ligand. By varying levels of IgE for priming, one may observe similar shifts in dose response to tropomyosin.

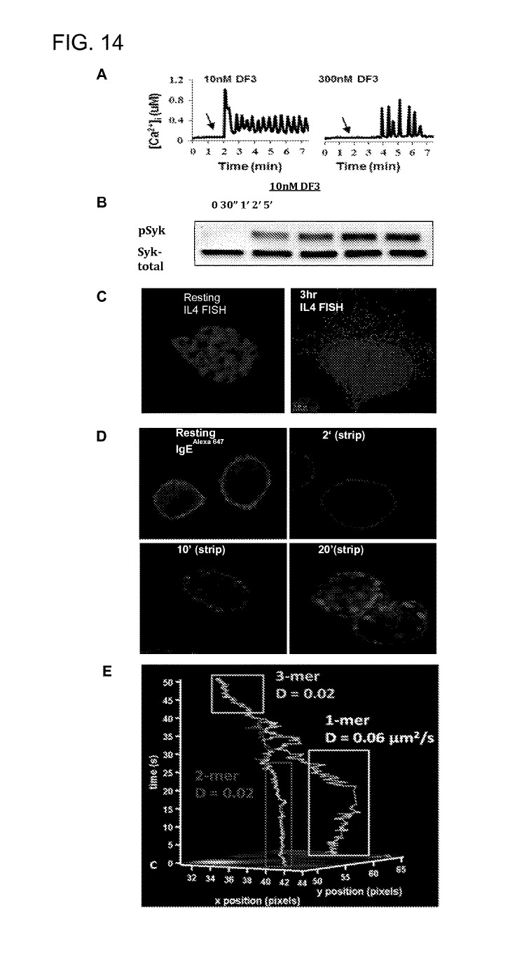

The clinical allergy field has relied almost exclusively on histamine release as a measure of mast cell and basophil functional responses for human allergens. While informative, this limited readout fails to account for the complex and branching FcεRI signaling pathway. An alternative approach uses other assays to characterize alternative measures of FcεRI signaling and regulation in response to tropomyosin (Pen a 1), and can apply these assays to the evaluation the response to other model allergens such as, for example, Ara h 2 and Jun a 1. Table 1 and FIG. 14 summarize the large number of assays already available in the laboratory for this characterization. Since signaling is initiated by Lyn-mediated phosphorylation of ITAM motifs, followed by recruitment of Syk kinase, one can evaluate the phosphorylation kinetics of these mediators on both positive and negative regulatory sites using proven reagents. Anti-phospho tyrosine antibodies allow one to evaluate the kinetics of immunoprecipitated FcεRI β and γ subunits. To evaluate binding kinetics of signaling partners, one can use proximity ligation assay (FIG. 8B). This is a robust single cell assay, since it detects protein-protein interactions that may be disrupted by detergents used in classical co-immunoprecipitation assays. Unlike western blotting methods, it also reports the subcellular location of the binding reaction. Many primary antibodies against FcεRI and its signaling proteins (e.g., Lyn, Syk, p85, PLCγ, LAT, etc.) have been validated. Hybridization between probes on secondary antibodies (e.g., DUOLINK secondary antibodies, Olink Biosciences, Uppsala, Sweden) serves as a template for amplification of fluorescent DNA probes at the site of contact. The assay can be readily applied to fixed samples over a time course of allergen stimulation. One can, for example, compare recruitment of positive and negative regulatory proteins (e.g., SHIP, Dok, SHP, CBP, and/or CBL), since this balance can govern the mast cell response across different ligand doses.

One can also use ratio imaging to measure calcium responses in Fura-2-loaded single cells. This sensitive assay captures oscillatory or early transient responses. Translocation of fluorescent-fusion proteins to the membrane (measured in TIRF) or into the nucleus (measured by confocal microscopy) are useful reporters of cell activity. One also can use single molecule fluorescent in-situ hybridization (smFISH) methods for fixed RBL cells to measure upregulation of cytokine RNA in response to ligand. The single cell, acid-stripping assay for receptor internalization is can be very informative, since FcεRI endocytosis can occur by either of two routes (Cleyrat, 2013, J Cell Sci 126:4913-4925) and can be affected by valency and dose.

Understanding the connection between allergen-mediated receptor aggregation and signaling responses may lead one to validate the computational models using microscopy assays. For example, one or more of three complementary methods can be used to validate a computational model: 1) Immunogold labeling of membrane sheets and electron microscopy, 2) Receptor “clustering” analysis using fluorescent ligand or IgE, and 3) Single particle tracking. FIG. 14E demonstrates how single particle tracking uniquely allows the observation of aggregation in real time. In this example, a pair of QD-tagged IgE receptors are already in a complex (2-mer) as imaging begins and move with correlated motion. At ˜40 seconds into image acquisition, a third QD-tagged receptor joins the complex and they diffuse together for 10 seconds. One also can capture the dissociation of individual receptors from an aggregate during imaging. After thousands of observations, it is possible to arrive at rate constants for these dissociation events. Variability in off-rates will reflect the range of IgE-allergen affinity in the serum (pooled and purified as a Ig fraction for each allergen group), providing a basis for evaluating complexities of individual immune responses.

A full analysis can thus include one or more of the following. One can, for example, prime hFcεRI on cultured mast cells with IgE purified from serum of atopic individuals. One can, for example, measure mast cell responses to authentic allergens and correlate these with quantitative measures of receptor aggregation and intracellular signaling. For example, calcium traces can be very informative: an elevated and sustained response is indicative of a robust signaling response, whereas oscillations are typical of weak signaling. The smFISH assay reports cytokine gene transcription more rapidly than classical ELISA methods and also has the advantage of reporting cell-to-cell variability. “Good” allergens can mobilize Syk and recruit lower levels of SHIP and other negative regulatory players. By comparing signaling generated by allergens with unique structure and valency, one can identify features that define a “successful” allergen.

To extend mechanistic studies of allergen-mediated crosslinking of FcεRI to human basophils isolated from allergic subjects, one can evaluate the typical percent of FcεRI occupied with allergen-specific IgE on the basophil cell surface and to determine the threshold of allergen needed for release of inflammatory mediators. Furthermore, one can determine if structurally distinct allergens induce similar or different responses as indicated by signaling readouts and comparative release of early and late phase mediators.

Unexpected exposure to food allergens is a common health issue. In the clinical setting, several large initiatives have attempted to determine if there is a safety threshold for severely allergic individuals. These studies have typically used oral challenges with minute to low doses.

Even when performed in hospitals, there is significant risk of adverse events. There is an important need to determine the threshold for allergen exposure at the cellular level, by evaluating responses outside the patient, and to link this to allergen-specific IgE levels.

Therefore, the responses of human primary cell to our test allergens can be characterized. There are likely to be differences in signaling output from the humanized cell lines and primary basophils, since humanized RBL cells are clonally derived and grow in defined conditions versus the polyclonal and changing in vivo environment of the human basophil. The hRBL studies allow one to prioritize these experiments, since performing the full range in primary cells would be prohibitively costly.

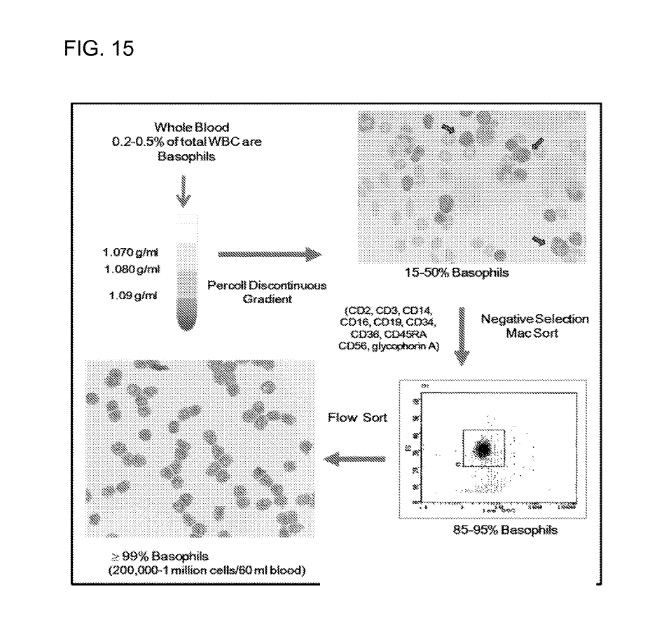

FIG. 15 outlines an exemplary routine protocol for basophil purification. One can use samples after the first centrifugation step. Basophils are readily identifiable in most single-cell imaging experiments or by scattering properties in flow assays. One may, however, use an alternative option of negative selection and flow sorting if very high purity is required. For example, basophils are the only possible source of histamine in peripheral blood but contaminating leukocytes can produce cytokines and other mediators.

There are several aspects of working with human basophils that require special consideration. First, the affinity of IgE-FcεRI is so tight that freshly isolated basophils can already be armed with a complex repertoire of IgE. Second, donors can erroneously self-report as allergic to a food or environmental allergen. Thus, one may wish to validate self-reported allergies by, for example, serum testing for allergen-specific IgE using, for example, a chemiluminescent assay such as the IMMULITE chemiluminescent assay (UNM-TriCore Reference Lab, Albuquerque, N. Mex.). Third, most atopic subjects are sensitized to many allergens. As a consequence, IgE against each test allergen can represent only a fraction of IgE decorating the basophil surface.

### Estimating Allergen-Specific IgE on Freshly Isolated Basophils.

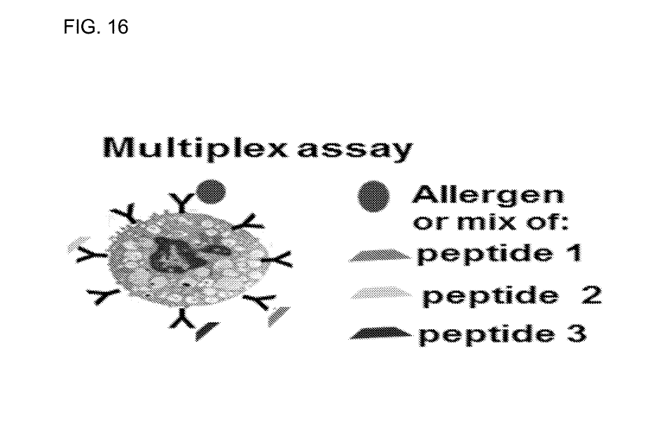

To evaluate the basophil triggering threshold (which may be different for the early and late phase responses), one needs a reasonable estimate of allergen-specific IgE on the cells from a donor or other subject. One may use, for example, a modified multiplex assay that takes advantage of multicolor flow cytometry facilities. As illustrated in FIG. 16, one can conjugate an allergen—either a native allergen or a recombinantly produced allergen—to FITC and determine the F/P (fluorophore/protein) ratio. Live basophils can be incubated with the fluorescent protein at 4° C. and binding measured by flow. To get a reasonable estimate of the levels of whole allergen bound, one can calibrate with, for example, FITC bead standards. While valency makes accurate quantitation challenging, one can compare relative values between donors by, for example, labeling replicate basophil samples from each donor with FITC-anti-IgE.

In addition to, or as an alternative to, evaluating total allergen specific IgE using the above assay, one can get a relative measure of IgE specific to the different linear epitopes on the allergen. To accomplish this, one can synthesize peptides with fluorescent tags of different colors, incubate them as a mix with basophils, and measure binding on multicolor flow cytometer such as, for example, the 15 color-LSRFortessa flow cytometer. A variation of this assay can include adding a large excess of dark peptide after reaching equilibrium with the fluorescent peptides. This protocol allows one to estimate the affinity of the donor's IgE for the linear epitope.

Correlation of Specific IgE Levels and Ligand Dose with Outcome Measures and Aggregation.

For each donor's or subject's basophils, one can perform dose response studies for release of inflammatory mediators and use single-cell imaging assays for signaling readouts. For example, calcium ratio imaging and smFISH for cytokine mRNA may be performed in particular. In addition, histamine and prostaglandin/leukotriene release assays are adaptable to a small volume, multi-well format. This can maximize the number of measures performed with basophils from each donor or other subject.

Armed with information about the prevalence of specific-IgE, estimates of linear epitope affinity, and/or structural predictions, one can generate useful predictions about how aggregate size, receptor occupancy, and/or receptor spacing set the threshold for human basophil “triggerability”. Thus, combined experimental and computational approaches may be used to estimate the minimum number and orientation of receptors that must be engaged for basophil activation.

Finally, one can use the data gathered as described above to design a recombinant immunotherapy reagents based upon the mechanistic understanding of allergen-IgE-FcεRI aggregation and mast cell/basophil signaling. Specifically, one can use a mutagenesis strategy to alter the spacing, affinity, and/or valency of allergens and determine the outcome on FcεRI activation.

One strategy for constructing a recombinant hypoallergen involves designing a fusion protein including an allergen-derived peptide and an unrelated carrier (such as, e.g., a viral coat protein) such that the formulation lacks allergenic activity. Other approaches include the synthesis of a short peptide, such as the peptide-based allergy vaccine strategy for cat dander allergies. In contrast, this disclosure describes using structural and mechanistic information about the allergen-IgE-FcεRI complex to reengineer the allergen. In some designs, one can retain as much of the original allergen as possible. One can create mixes of recombinant molecules that are each mutated at different points in the structure to disrupt the spacing between linear or conformational epitopes and/or to lower affinity. These mutations can be specifically designed to limit FcεRI triggering when administered, decreasing—and in some cases even eliminating-allergic responses, while retaining antigenicity that can be superior to other approaches that rely solely on short peptides or are extensive modifications of the allergen.

In previous development of hypoallergens and peptide vaccines, the principal screening criteria has been poor degranulation response of humanized RBL cells. However, the FcεRI signaling pathway is highly branched and there is strong evidence that the three different arms of the pathways leading to 1) degranulation, 2) cytokine production, and 3) leukotriene/prostaglandin release have distinct sensitivities to ligand dose and amount of specific IgE. In addition, these arms can be differentially influenced by other signaling pathways that tune primary cells in vivo for responsiveness. Degranulation correlates poorly, however, with cytokine production in human basophils. Moreover, differences can exist in the extent to which degranulation and cytokine responses are inhibited after crosslinking residual IgE on basophils isolated from volunteer subjects on omalizumab therapy. Thus, this disclosure describes testing in human basophils isolated from allergic subjects, where the levels of allergen-specific IgE, receptors, and FcεRI signaling partners are more relevant to the clinical setting.

### Mutagenesis of IgE Epitopes on Allergens.

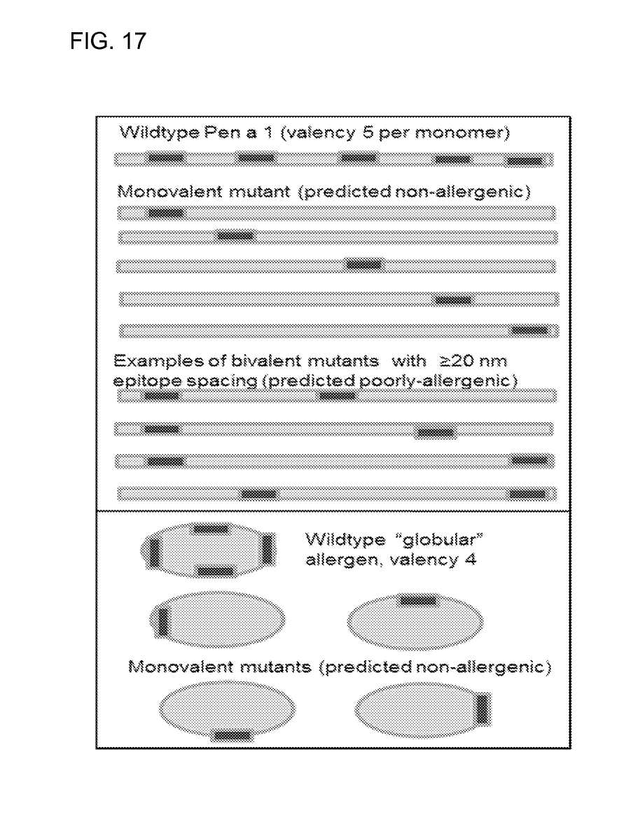

FIG. 17 illustrates how one can produce recombinant allergens that are either effectively monovalent or have low valency, but with poor antigenicity based upon retaining only those pairs of epitopes that exceed the crosslinking requirements for FcεRI activation. This strategy is based upon the observation that spacing between haptens in stiff DNA-based bivalent ligands for IgE-FcεRI crosslinking was crucial for signal initiation. This spacing was optimal at approximately 5 nm and progressively poorer as the distance increased. The artificial DF3 ligand meets this requirement, with center-to-center distances ranging from 3.4-6 nm (due to predicted flexibility of short peptide linkers). Table 2 reports the predicted spacing between neighboring epitopes on the selected model allergens to range from 0.9 to 10.3 nm (9.2-102.7 Å) (SDAP database). Thus, some combinations of epitopes might be too close to be effective at crosslinking FcεRI. For Pen a 1, the most distant two epitopes are >30 nm apart and may be too far for optimal crosslinking.

TABLE 2
Epitope spacing for test allergens

| | | Minimum | |
|---|---|---|---|
| Allergen | Epitopes | spacing (Å) | Maximum spacing (Å) |
| Ara h 2 | 6 | 9.2 | 33.9 |
| Jun a 1 | 4 | 17.4 | 38.8 |
| Pen a 1 | 18 (in five clusters, | 67.0 | 102.7 |
| | boxes, top of FIG. 17) | | |

For the shrimp tropomyosin, Pen a 1, amino acid substitutions in each of the five epitopes that result in hypoallergenicity are known. Instead of targeting all five epitopes at once, however, one can selectively produce monovalent and bivalent forms that retain only one or two unmodified IgE binding epitopes in each mutant protein. Structural analysis suggests that the tropomyosin molecule is sufficiently rigid that it will not fold back upon itself, bringing distant epitopes together.

Based upon IgE-binding maps of the more globular Ara h 2 and Jun a 1 proteins, one can use structural analysis to target the central residues of known linear or conformational epitopes. For the linear epitopes, one option to shorten the screening process is to synthesize short peptides with substituted amino acids and use a competitive assay. With this assay, one can identify peptides that fail to compete with whole antigen for binding to patient-derived IgE. For Ara h 2, one can evaluate a naturally occurring polymorphism in the immunodominant epitope 7, which substitutes a threonine for a serine at position 73. This polymorphism appeared to markedly reduce IgE-binding activity.

Once one has mapped the individual mutations needed to decrease binding, one can use the monovalent mutants for cell activation assays. Based bivalent reagents can have strict distance and/or flexibility requirements, some bivalent mutants may be hypoallergenic. Some mutations may lead to a disruption of secondary or tertiary structure. As a precaution, one can evaluate a recombinant allergen in solution using, for example, circular dichroism techniques.

The choice of system (bacterial, insect cells, yeast, or plant) used to produce a recombinant allergen may influence antigenicity of the allergen due to glycosylation and/or other post-translational modifications. A person of ordinary skill in the art can select an appropriate system for producing a recombinant allergen using, for example, the IgE reactivity of wildtype allergen as a control.

One can design the recombinant allergen to include an affinity tag such as, for example, a His-tags for rapid purification.

### Testing for Impact of Allergen Mutations on Binding to IgE, FcεRI Aggregation and Downstream Responses.

Initial testing of modified allergens can be performed in humanized RBL cells, primed with the IgE purified and pooled from the serum of allergen-specific donors. One can then test the recombinant allergen with freshly isolated basophils from allergic subjects. A recombinant allergen may be evaluated using, for example, any one or more of the single cell assays described above such as, for example, the single cell assays relating to calcium ratio imaging and/or cytokine mRNA. Alternatively, or additionally, a recombinant allergen may be evaluated using any of the multi-well assays described above such as, for example, those relating to histamine and/or prostaglandin/leukotriene release. In some cases, when evaluating a recombinant allergen, anti-IgE and wildtype allergen challenges can be performed on the same preparation of donor/subject cells in order to control for the possibility that some donors/subjects naturally cycle to non-releaser status while on study.

Compositions that include a recombinant allergen can include a single recombinant allergen, a mixture of a plurality of recombinant allergens, or a mixture of one or more recombinant allergens with additional components such as, for example, additional proteins and/or adjuvants.

One can use imaging assays to evaluate aggregation and ligand dissociation using a recombinant allergen. For example, disrupted epitope spacing may alone be insufficient to optimize some hypoallergens. In addition to changing valency and epitope spacing, one may design a recombinant allergen that separate secondary features in allergens using an unstructured linker that increase overall flexibility. Such linkers may, without wishing to be bound by any particular theory, behave similarly to PEG-based trivalent ligands for FcεRI activation, where the “floppy” nature of PEG groups lowered degranulation responses even under conditions where epitope distance was predicted to be permissive for signaling. Also, affinity of IgE for recombinant allergen epitopes may vary across the basophil samples from our donor pool.

Affinity estimation can initially come from the fluorescent peptide flow-based assay (FIG. 16) but the clearest evidence for effects on FcεRI aggregate stability may come from multicolor single particle tracking where it is possible to observe aggregate dissociation in real time (FIG. 14E). The same methods used to characterize wildtype allergens, described above, can be used to interpret test results using recombinant allergens. For example, one can use the computational approaches described above to evaluate changes in aggregation (i.e., size, composition, orientation) induced by any recombinant allergen that has a valency greater than 1.

Thus, this disclosure describes a method for designing an engineered hypoallergen. Generally, structural requirements for FcεRI aggregation and signaling can provide guidelines for designing recombinant hypoallergens. Furthermore, we show the feasibility of using mixtures of minimally modified hypoallergens for improved immunotherapy. Taken on the whole, the mixtures can retain important B cell and T cell epitopes thought to be involved in providing clinically-relevant activity.

While described below in the context of a model food allergen, Pen a 1, the methods described above and the recombinant allergens developed using the methods can involve any suitable allergen including, for example, the peanut allergen Ara h 2, the environmental allergen Jun a 1), the feline allergen Fel d 1 (Felis domesticus), and/or pollen allergens similar in structure to quadravalent Par j 1 (Parietaria judaica).

Generally, a recombinant allergen includes at least one amino acid modification compared to a corresponding wildtype allergen so that the recombinant allergen—a hypoallergen—(a) binds to IgE that specifically binds to the allergen and (b) induces release of histamine from basophils to a degree less than the wildtype (e.g., native) allergen. As used herein, “specific” and variations thereof refer to having a differential or a non-general (i.e., non-specific) affinity, to any degree, for a particular target. Thus, binding of the recombinant allergen by the IgE does not diminish the IgE specificity with regard to the native allergen. As used herein, the term “amino acid modification” refers, collectively, to any amino acid addition, amino acid deletion, and/or any amino acid substitution compared to the wildtype or other native form of the allergen.

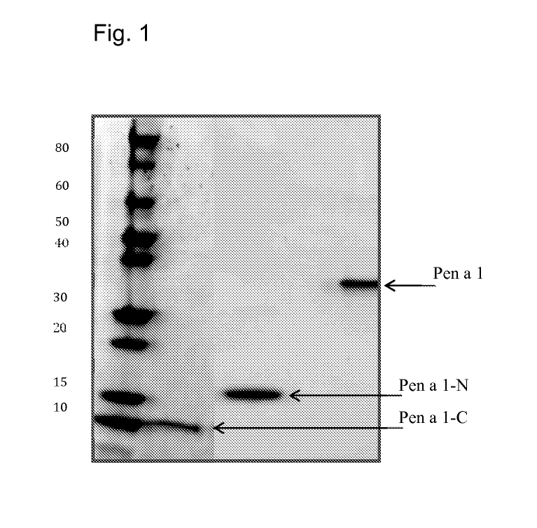

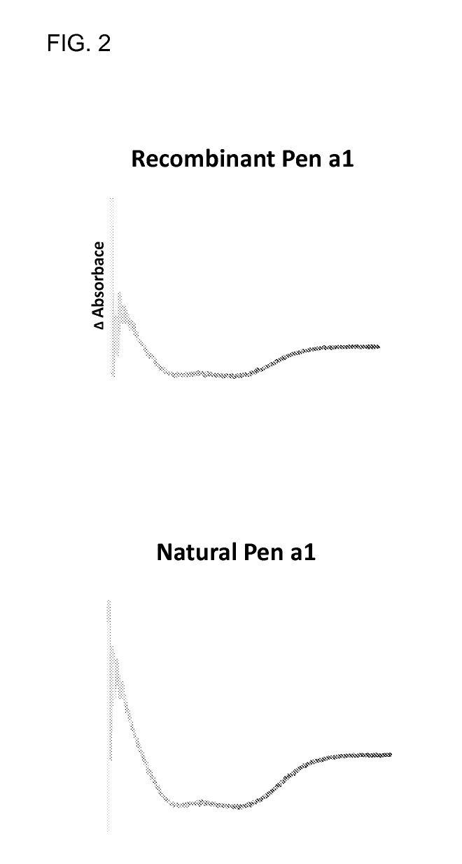

One exemplary designed hypoallergen involves truncated fragments corresponding to the N-terminal and C-terminal ends of the primary shrimp allergen, Pen a 1 (SEQ ID NO:1, tropomyosin derived from P. aztecus, now Farfantepeneaus aztecus). FIG. 1 shows expression of recombinant Pen a 1 and its fragments in E. coli. FIG. 2 shows that recombinant Pen a 1 has intact coiled coil structure, similar to natural Pen a 1 using CD (circular dichroism) spectroscopy.

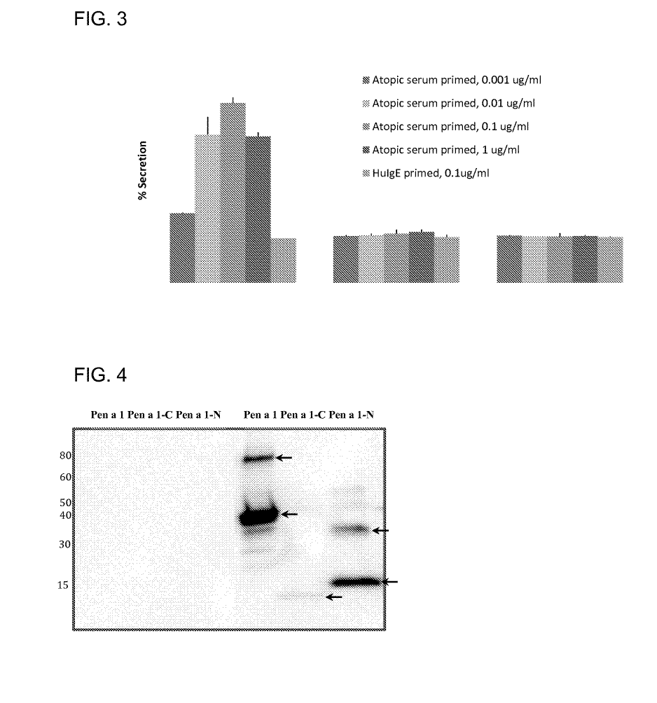

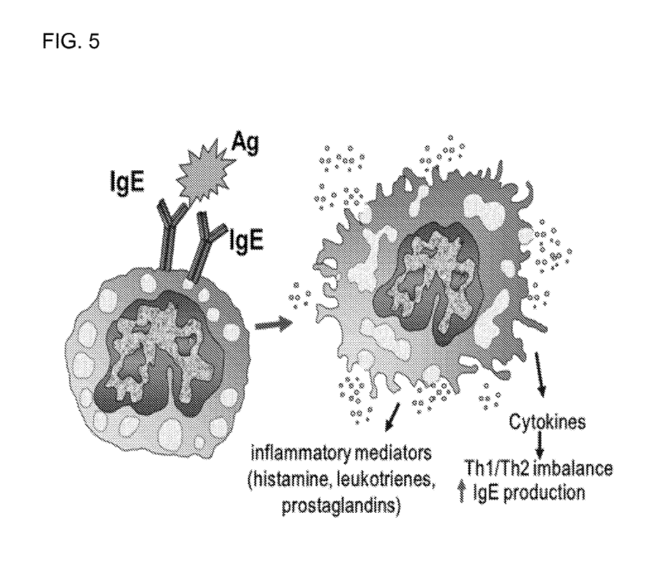

Pen a 1-N(N-terminal fragment, amino acids 1-79 of SEQ ID NO:1, also referred to as FR1) and Pen a 1-C (C-terminal fragment; amino acids 224-284 of SEQ ID NO:1, also referred to as FR5) bind IgE from serum of shrimp-allergic donors, but do not trigger histamine release. FIG. 3 and FIG. 4. This is evidence that the fragments fail to crosslink IgE-FcεRI complexes on the cell surface and/or create small aggregates that are incompetent to signal. Thus, recombinant Pen a 1 allergens (e.g., C-terminal or N-terminal fragments) can be used as hypoallergens for improved immunotherapy based on their ability to bind to and decrease the activity of IgE by creating monovalent or sub-optimal crosslinking conditions. With reduced—and in some cases, absence of—IgE receptor activation that results from the recombinant allergen binding to the IgE, mast cells and basophils exhibit decreased degranulate during treatment with the recombinant allergen, decreasing the risk, extent, and/or severity of adverse events associated with exposure to the wildtype allergen.

Additional exemplary embodiments include additional fragments of Pen a 1 such as, for example, amino acids 68-127 of SEQ ID NO:1 (FR2), amino acids 121-181 of SEQ ID NO:1 (FR3), and amino acids 172-236 of SEQ ID NO:1 (FR4).

### Quantification of Dose-Dependent Pen a 1-Mediated Degranulation Responses and Corresponding FcεRI Occupancy

Rat basophilic leukemia cells modified for exclusive expression of the human α-subunit of FcεRI (hRBLrko) were used for these studies. To accomplish this, the rat FcεRI α-subunit was knocked out in RBL-2H3 cells using CRISPR technology, followed by transfection with an expression vector for the human FcεRI α-subunit. The stably transfected cells are thus unique in that there are no rodent FcεRI α tetrameric receptors to compete with the human α-bearing FcεRI tetramers for coupling with signaling partners.

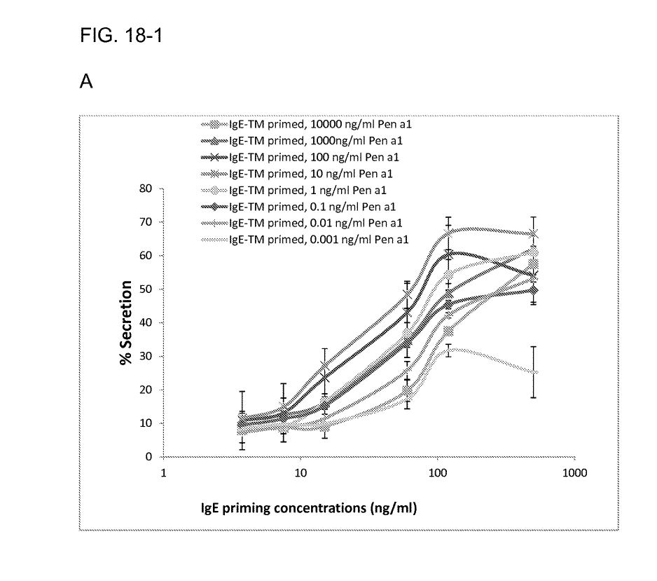

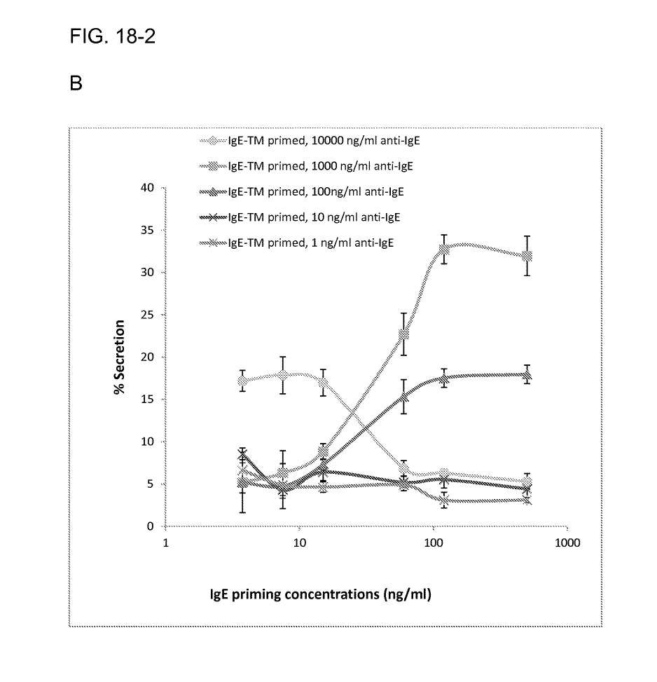

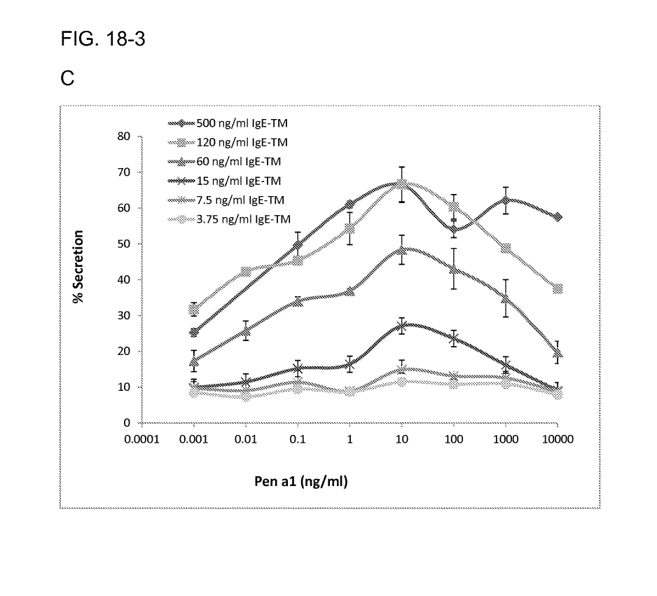

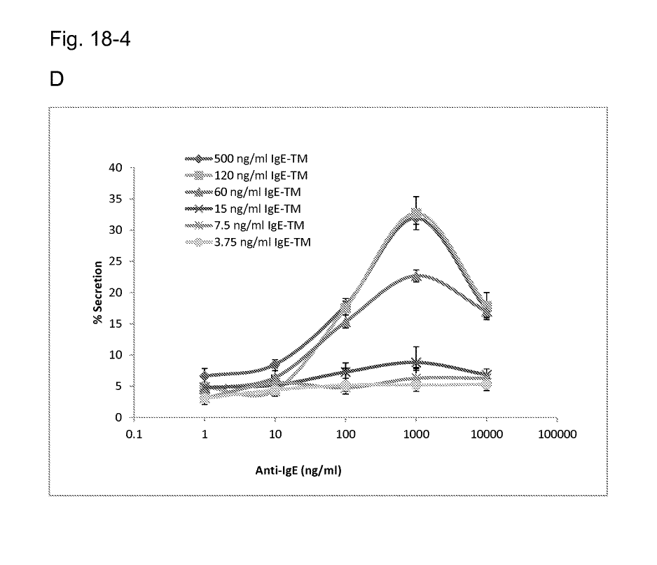

Cells were primed with a range of concentrations of Pen a 1-specific IgE (IgE^{pena1}) that was affinity purified from shrimp-reactive plasma as described in Example 1. Cells were then activated with increasing doses of recombinant Pen a 1 (rPen a 1) or anti-IgE (as a positive control) to measure degranulation responses (FIG. 18). The data are plotted two ways: as a function of IgE priming conditions (FIGS. 18A and B) or as a function of crosslinker concentration (FIGS. 18C and D). These two presentations of the same data sets illustrate that, while there is a saturating concentration of IgE that reaches a plateau at approximately 500 ng/ml (FIGS. 18A and B), the typical bell-shaped secretory response, seen with structurally defined ligands as well as natural allergens, is reproduced in the hRBLrko cells challenged with Pen a 1 (FIGS. 18C and D; SEQ ID NO:1).

An assay using fluorescent IgE and flow cytometry (Table 1) was developed to correlate secretory responses with the number of allergen-specific FcεRI bound. The minimal IgE priming conditions for stimulating secretion occurred after two hours exposure to IgE^{pena1} concentrations of 15 ng/ml (6.25 kU/L using conversion values as in Dolen W., 2003, Allergy 58:717-723). Placing these values within the range of positive IMMUNOCAP scores for the allergic subjects that we evaluated, a number of subjects had circulating shrimp-specific IgE levels above this threshold. Importantly, these data suggests that the minimum number of FcεRI that need to be engaged for measurable response is approximately 300 FcεRI (FIG. 18C, 15 ng/ml IgE). To achieve maximal secretion, less than 3000 FcεRI need to be engaged by Pen a 1-mediated crosslinking (FIG. 18C, 120 ng/ml IgE). Note that rPen a 1 causes significant mast cell degranulation responses at just 1 pg/ml, stimulating 20% secretion when less than 2% of total receptors are occupied (FIG. 18C, 60 ng/ml IgE). The optimal dose of rPen a 1 was 10 ng/ml, which did not shift with increasing levels of priming IgE^{pena1}.

Simulated Gastric Digestion Produces rPen a 1 IgE-Binding Peptides with Modestly Attenuated Mediator Release

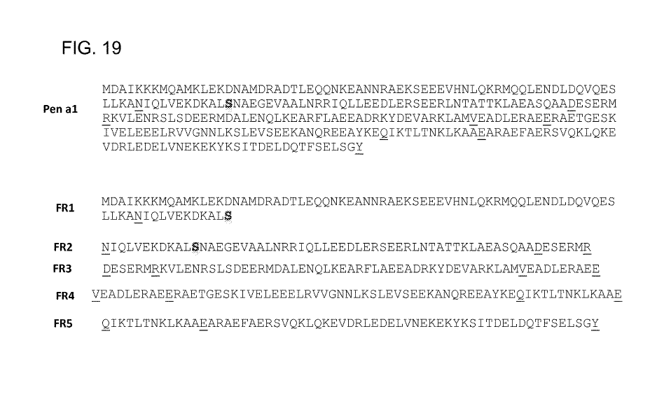

In vitro digestion of rPen a 1 with pepsin in SGF was analyzed using SDS-PAGE (FIG. 19A). After 10 minutes of digestion with 0.1 μg/ml pepsin, faint fragments around 33 and 17 kDa appeared. With an increase in pepsin concentration up to 3 μg/ml, 33 kDa fragment decreased and 17 kDa fragment increased dose-dependently. Only upon treatment with higher concentrations of pepsin (10 μg/ml) was rPen a 1 robustly digested to generate small peptides (˜10-20 kDa). Immunoblot analysis of digested rPen a 1 (FIG. 19B) revealed that IgE from the serum of atopic patient readily recognized both the 33 and 17 kDa fragments. With 10 μg/ml pepsin treatment, two smaller fragments between 15-20 kDa were recognized by IgE. Although modest digestion of rPen a 1 did not reduce reactivity to IgE, it progressively decreased the ability of rPen a 1-derived peptides to cause effector cell mediator responses. Stimulation of atopic serum primed hRBL cells with digested mixtures caused dose-dependent reduction in cellular secretory responses (FIG. 19C). While the degranulation response decreased, concentration of smaller 17 kDa fragment increased and larger 33 kDa fragment decreased, with increasing pepsin concentrations used for digestion (FIGS. 19D and E).

### Design and Characterization of Pen a 1 Hypoallergens

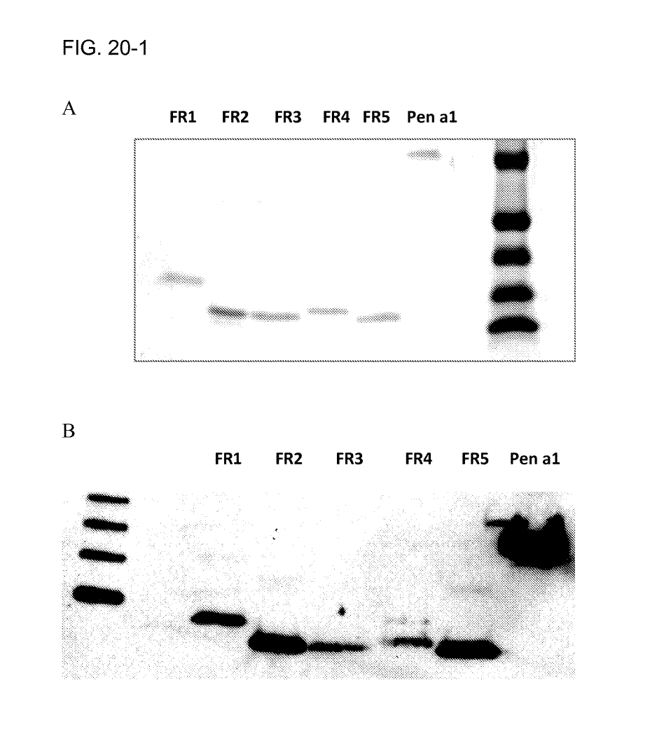

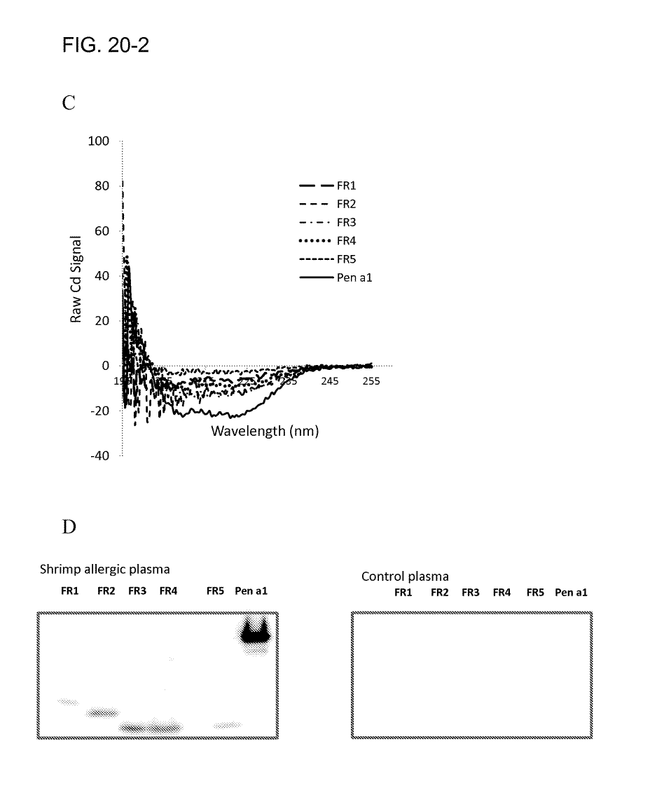

Five sequential overlapping fragments (FR1¹⁻⁷⁹, FR2⁶⁸⁻¹²⁷, FR3¹²¹⁻¹⁸¹, FR4¹⁷²⁻²³⁶, FR5²²⁴⁻²⁸⁴, FIG. 20A) were designed. Each fragment was 60-79 amino acids long and included either a single predominant IgE binding epitope or closely aligned epitopes. These fragments were designed to cover the entire sequence of Pen a1, retaining known IgE and T-cell epitopes.

Fragments had an overlap of 7-12 amino acids. A C-terminal His tag was linked to Pen a 1 and each fragment. Recombinant proteins were expressed in E. coli, purified using Ni-NTA columns and separated using size exclusion chromatography to yield a single band on SDS-PAGE and identified with an anti-His antibody on a western blot.

Secondary structure of recombinant full-length Pen a 1 (SEQ ID NO:1) and fragments was evaluated using circular dichroism (CD). rPen a 1 had a typical α-helical structure based on the characteristic minima at 208 and 222 nm and maxima at 193 nm. The Pen a 1 fragments exhibited varying amounts of α-helical content that was significantly lower than the α-helical content seen in rPen a1.

The binding of IgE from shrimp allergic plasma was confirmed by western blotting, as shown in FIG. 20E. While rPen a 1 had maximal binding, FR1, FR2, and FR3 bound the most among the fragments and FR4 had the least recognition. None of the fragments or the whole Pen a1 bound IgE from control serum.

### Recombinant Pen a 1 Hypoallergen Fragments Fail to Stimulate Significant Degranulation and Act as Competitive Inhibitors for Intact Pen a 1

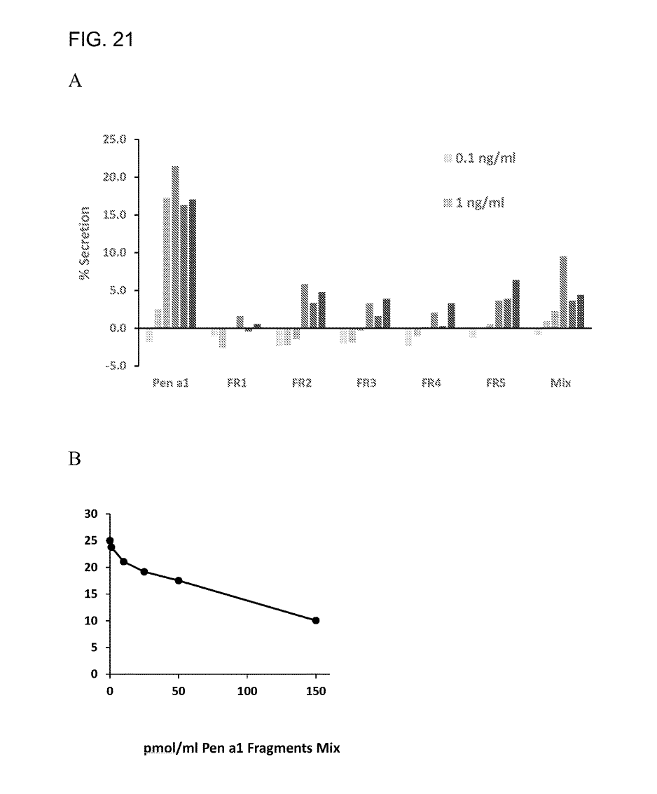

Humanized hRBLrko cells were sensitized with IgE by incubation with serum from a shrimp-allergic subject, followed by a challenge with increasing concentrations of either whole rPen a 1, each of the five fragments alone or as mixture (FIG. 21A). Minimal secretory response to all five fragments was observed, even when challenging cells the highest dose of 1 μg/ml. When all five fragments where pooled, only very small secretory responses to 100 ng/ml (<10%) were observed.

Next, the ability of Pen a1 fragments to compete with intact Pen a1 for mediator release was tested in hRBLrko cells. In FIG. 21B, sensitized cells were incubated first for five minutes with 1 μg/ml Pen a 1, followed by addition of pooled fragments at defined concentrations for 25 minutes. Addition of the fragments lowered the over secretory response in a dose dependent manner. These data show that the recombinant fragments serve as competitive inhibitors for intact allergen.

This disclosure therefore characterizes the relationship between Pen a 1 and IgE^{pena1} priming, enabling one to determine the number of FcεRI occupied and cellular response. While measurable degranulation responses were seen with only a few hundred receptors engaged on the cell surface, robust responses were obtained with concentrations of priming IgE that engage less than 3000 (or 4% of total) receptors. Adverse reactions like anaphylaxis can occur with only 0.1-20% of receptors occupied with allergen specific IgE on cell surface of mast cells and basophils. These results demonstrate that defining the mechanisms of initiation and propagation of allergic reactions can aid in predicting the likelihood of an allergic response based on circulating IgE levels and food allergen concentrations.

Immunotherapy for food allergies is still not an established treatment option, given the risk of adverse reactions often seen with food like peanuts (Nelson et al., 1997, J Allergy Clin Immunol 99:744-751). Conventional strategies for designing immunotherapy for allergy typically target the design of hypoallergens that show reduced or abolished IgE binding while retaining T cell epitopes for retention of immunogenicity. One of the methodologies for reducing IgE binding includes destroying the structural conformation of the allergen through with heat-induced denaturation or in vivo digestion. The structure of Pen a 1 is, however, extremely heat-stable and is not altered even after boiling of shrimp (Lehrer et al., 1990, J Allergy Clin Immunol 85:1005-1013), and fully retains its allergenicity post heat treatment (Leung et al., 1994, J Allergy Clin Immunol 94:882-890).

To evaluate the allergenicity of Pen a 1 after digestion, the IgE binding and effector cell activation of digested Pen a 1 fragments were characterized. There was a dose-dependent reduction in cellular mediator release as measured by functional secretion assays. These results suggest that reduced allergenic potency can be obtained by lowering allergen valency while retaining IgE binding. Although digestion of Pen a 1 reduced shrimp allergenicity in this study, fragments of tropomyosin produced by digested shrimp in allergic individuals still triggered an allergic reaction. This must be attributed to the variability in digestion from person to person that results from varying pepsin-to-substrate ratio.

The Pen a 1 fragments described herein were thus designed to reduce valency and allergenicity. The designed fragments are longer than fragments that result from digestion with pepsin. The fragments were evaluated for IgE binding and effector cell responses. Similar to pepsin-digested Pen a 1, the fragments retained the ability to bind IgE. However, the fragments induced very weak secretory responses in hRBL cells, even at high concentrations and when pooled together for stimulating cells. Moreover, the fragments competed with intact Pen a 1 for cell activation as seen with inhibition assays in hRBL cells. Reduced mediator release upon cellular binding of fragments combined with their ability to compete off intact Pen a 1 is supportive of the use of the fragments as hypoallergens in a therapeutic strategy for treating shrimp allergy.

While occasionally described herein in the context of an exemplary embodiment in which the hypoallergen is derived from Pen a 1 from Farfantepeneaus aztecus, a hypoallergen as described herein can be derived from any other suitable allergen. Exemplary alternative embodiments can include a hypoallergen derived from Pan a 1 from other species of shellfish, a peanut allergen (e.g., Ara h 2 from Arachi hypogaea), pollen allergen (e.g., Par j 1 of Parietaria Judaica), cat allergen (e.g., Fel d 1 from Felis domesticus), or an environmental allergen (e.g., Jun a 1 from Juniperus ashei). As used herein, the term “derived from” refers to a polypeptide that includes at least one amino acid modification compared to a native (e.g., wild-type) form of the allergen. Thus a hypoallergen can include, for example, a truncated form compared to the native form of the allergen. In some embodiments, a truncated hypoallergen can include at least one IgE binding epitope. In some cases, a truncated hypoallergen can include more than one IgE binding epitope.

A hypoallergen that reflects a truncated form of a native allergen can have a minimum length of at least 30 amino acids such as, for example, at least 35 amino acids, at least 40 amino acids, at least 45 amino acids, at least 50 amino acids, at least 51 amino acids, at least 52 amino acids, at least 53 amino acids, at least 54 amino acids, at least 55 amino acids, at least 56 amino acids, at least 57 amino acids, at least 58 amino acids, at least 59 amino acids, at least 60 amino acids, at least 61 amino acids, at least 62 amino acids, at least 63 amino acids, at least 64 amino acids, at least 65 amino acids, at least 70 amino acids, or at least 75 amino acids. A hypoallergen that reflects a truncated form of a native allergen can have a maximum length of no more than 100 amino acids such as, for example, no more than 95 amino acids, no more than 90 amino acids, no more than 85 amino acids, no more than 84 amino acids, no more than 83 amino acids at least 82 amino acids, at least 81 amino acids, at least 80 amino acids, at least 79 amino acids, at least 78 amino acids, at least 77 amino acids, at least 76 amino acids, at least 75 amino acids, at least 70 amino acids, or at least 65 amino acids. In some cases, the length of a truncated hypoallergen can be expressed as a range having endpoints defined by any minimum length described above and any maximum length described above that is greater than the minimum length. For example, a hypoallergen can have a length of from 60-79 amino acids.

In certain embodiments, a truncated hypoallergen can have a length of 60-79 amino acids and includes at least one IgE binding epitope.

Thus, this disclosure describes pharmaceutical compositions and methods for treating allergy. As used herein, “treat” or variations thereof refer to reducing, limiting progression, ameliorating, or resolving, to any extent, the symptoms or signs of allergy. As used herein, “ameliorate” refers to any reduction in the extent, severity, frequency, and/or likelihood of a symptom or clinical sign of allergy. A “treatment” may be therapeutic or prophylactic. “Therapeutic” and variations thereof refer to a treatment that ameliorates one or more existing symptoms or clinical signs of allergy. “Prophylactic” and variations thereof refer to a treatment that limits, to any extent, the development and/or appearance of a symptom or clinical sign of allergy. Generally, a “therapeutic” treatment is initiated after the subject is exposed to an allergen, while “prophylactic” treatment is initiated before a subject is exposed to an allergen.

As used herein, “symptom” refers to any subjective evidence of allergy. Exemplary symptoms of allergy include itching (e.g., skin, eyes, ears, nose and/or throat), postnasal drip, shortness of breath, and/or chest tightness. As used herein, “sign” or “clinical sign” refers to an objective physical finding of allergy relating capable of being found by one other than the patient. Exemplary clinical signs of allergy include skin redness, rash, hives, wheezing, dyspnea, cough, sneezing, runny and/or stuffy, nausea, vomiting, diarrhea, low blood pressure, increased heart rate, fainting, behavioral changes, anaphylaxis, and/or swelling of the lips, tongue, nose, or throat.

Prophylactic treatment may be administered to a subject “at risk” of developing an allergic reaction to an allergen. In some cases, an “at risk” subject can be an individual known to manifest symptoms or clinical signs of allergy to an allergen and are subject to an increase in the likelihood of being exposed to the allergen. In other cases, a subject “at risk” of developing an allergic reaction to an allergen may not have manifested symptoms or clinical signs of allergy to an allergen, but may possess one or more risk factors of allergy to the allergen such as, for example, genetic predisposition, ancestry, age, and/or sex.

Thus, the hypoallergens described herein may be formulated with a pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with hypoallergen without causing any undesirable biological effects or interacting in a deleterious manner with any of the other components of the pharmaceutical composition in which it is contained.

A hypoallergen may therefore be formulated into a pharmaceutical composition. The pharmaceutical composition may be formulated in a variety of forms adapted to a preferred route of administration. Thus, a composition can be administered via known routes including, for example, oral, parenteral (e.g., intradermal, sublingual, transcutaneous, subcutaneous, intramuscular, intravenous, intraperitoneal, etc.), or topical (e.g., intranasal, intrapulmonary, intravaginal, intradermal, transcutaneous, rectally, etc.). A composition also can be administered as a sustained or delayed release formulation.

Thus, a hypoallergen may be provided in any suitable form including but not limited to a solution, a suspension, a tablet, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a flavoring, a moisturizer, a thickener, and the like.

A formulation may be conveniently presented in unit dosage form and may be prepared by methods well known in the art of pharmacy. Methods of preparing a composition with a pharmaceutically acceptable carrier include the step of bringing the hypoallergen into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

The amount of hypoallergen administered can vary depending on various factors including, but not limited to, the specific hypoallergen being administered, the weight, and/or age of the subject, and/or the route of administration. Thus, the absolute weight of hypoallergen included in a given unit dosage form can vary widely, and depends upon factors such as the age, and weight of the subject, and/or the method of administration. Accordingly, it is not practical to set forth generally the amount of hypoallergen that will be effective for all possible applications.

Those of ordinary skill in the art, however, can readily determine the appropriate amount with due consideration of such factors.

In some embodiments, the method can include administering sufficient hypoallergen to provide an optimal maintenance dose of approximately 5 μg to 20 μg to the subject, although in some embodiments the methods may be performed by administering hypoallergen in a dose outside this range. Many current therapeutic preparations for allergen immunotherapy are based upon use of allergen extracts, the exact composition of which is not always known. In contrast, the pharmaceutical compositions described herein that include a recombinant hypoallergen can have a uniform, standardized composition that can provide for improved safety compared to conventional allergen extract compositions.

In general, an outcome (e.g., decreased basophil histamine release or skin test) can be used to establish appropriate doses and schedule of administration of hypoallergen to be used. Although 5 μg to 20 g of native allergens can be effective and safe for treating, e.g., allergic rhinitis, a composition that includes a recombinant hypoallergen may be effective when administered in a lower amount. One can determine a dose that decreases the subject's allergen-induced basophil histamine release, determine the duration of that response, and then determine the frequency of administration that will maintain the desired effect.

In some embodiments, hypoallergen may be administered, for example, from a single dose to multiple doses per week, although in some embodiments the method can be performed by administering hypoallergen at a frequency outside this range. In certain embodiments, hypoallergen may be administered subcutaneously from about once per month to cluster regimens (e.g., two or more injections per visit on nonconsecutive days) to rush therapy (e.g., several doses administered over the course of hours on consecutive days). For sublingual immunotherapy (SLIT) formulations, the initial dose may be administered under the supervision of a physician to monitor for adverse events. In all of the above possible regimes, an exemplary initial outcome can include a decrease of allergen-induced basophil histamine release or skin test, and secondly clinical effectiveness.

In the preceding description, the term “and/or” means one or all of the listed elements or a combination of any two or more of the listed elements; the terms “comprises” and variations thereof do not have a limiting meaning where these terms appear in the description and claims; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is further illustrated by the following examples. The particular examples, materials, amounts, and procedures are to be interpreted broadly in accordance with the scope and spirit of the invention as set forth herein.

### EXAMPLES

### Cell Isolation and Histamine Release Assays.

Histamine release was measure as described previously (Kepley et al., 2000, J Immunol 165:5913-5920; Youssef et al., 2002, J Allergy Clin Immunol 110:366-373). Briefly, Percoll gradient centrifugation was used to prepare basophil-enriched cell populations (1-55% basophils) from 54 ml anticoagulated blood. Enriched cell fractions were activated by incubating with anti-IgE, Pen a1, or the fragments at 37° C. Spontaneous degranulation was measured by adding Hanks only to the cells. Calcium ionophore A23187 was used as an internal positive control. Cells were activated for 30 minutes, after which reactions were terminated by adding excess ice-cold HBSS. Post centrifugation, histamine in cell supernatants was measured by ELISA (Genway Biotech, Inc., San Diego, Calif.) according to the manufacturer's instructions. Total histamine was measured in supernatants generated by lysis of identical cell aliquots using three freeze-thaw cycles. The net FcεRI-mediated histamine released in response to ligand was expressed as a percentage of total histamine release after subtraction of spontaneous histamine release.

### Study Subjects

Individuals with shrimp allergy were recruited based on self-reporting. Shrimp allergy was confirmed by IMMUNOCAP assay (Phadia AB, Uppsala, Sweden) performed at Tricore Reference Laboratories (Albuquerque, N. Mex.), which determines IgE reactivity with crude shrimp extract. Values >0.35 kU/L were considered positive. Subjects that were positive for shrimp allergy as well as the control subjects were clinically assessed. For some studies, serum of shrimp allergic and control individuals was directly purchased from PlasmaLab International (Everett, Wash.). Serum samples were stored at −20° C.

### Purification of IgE^{pena1}

Pen a 1-specific IgE was purified using the EPIMAX affinity purification kit (Abcam, Cambridge, Mass.) according to the manufacturer's instructions. IgG was removed by incubating elution fractions with protein A/G beads (Invitrogen, Grand Island, N.Y.) for two hours. Absence of IgG in fractions was confirmed by immunoblotting with anti-IgG HRP (Invitrogen, Grand Island, N.Y.).

CRISPR-Edited hRBLrko Cells

Cas9-mediated DNA cleavage was used to knock out the endogenous rat α-subunit of RBL-2H3 cells. A highly specific gRNA, directed against the first exon of the rat FcεRIα genomic sequence, was designed using the CRISPR Design portal (Massachusetts Institute of Technology, Cambridge, Mass.) and then sub-cloned into the PX458 vector (plasmid #48138, Addgene, Cambridge, Mass.) for simultaneous expression of the gRNA, WT Cas9, and a GFP reporter. For the sub-cloning, two partially complementary oligonucleotides were ordered from Integrated DNA Technologies, Inc. (Coralville, Iowa) and assembled by PCR. Gel purified PCR products were cloned into BbsI-digested PX458 using Gibson Assembly (New England Biolabs, Inc., Ipswich, Mass., USA) following the manufacturer's specifications. The final plasmid, after cloning and sequencing, was used to transfect RBL-2H3 cells using the AMAXA system (Lonza Group, Ltd., Basel, Switzerland).

### Cloning

Pen a 1 cDNA (GenBank: DQ151457.1 Farfantepenaeus aztecus) was synthesized by Genewiz, Inc. (South Plainfield, N.J.). Five fragments of Pen a1 and Pen a1 were amplified using PCR and cloned into pET101 vector (Invitrogen, Grand Island, N.Y.). Primers were synthesized by Integrated DNA Technologies, Inc. (Coralville, Iowa). All constructs were expanded in ONE-SHOT TOP10 chemically competent E. coli (Invitrogen, Grand Island, N.Y.) and plasmid DNA was isolated using NUCLEOSPIN plasmid kits (Macherey-Nagel GmBH & Co., Duren, Germany). Sequencing was performed using Genewiz, Inc. (South Plainfield, N.J.). Primers used for amplification are as follows:

F1:
(SEQ ID NO: 2)
5′-CACCATGGACGCCATCAAGAAGAAGATGC-3′;
R1:
(SEQ ID NO: 3)
5′-AGAGAGGGCCTTGTCCTTGTC-3′
F2:
(SEQ ID NO: 4)
5′-CACCATGAACATCCAGCTTGTGGAGAA-3′
R2:
(SEQ ID NO: 5)
5′-GTTCTCGAGCACCTTGCGCAT-3′
F3:
(SEQ ID NO: 6)
5′-CACCATGAAGGTGCTCGAGAACC-3
R3:
(SEQ ID NO: 7)
5′-CTCCTCAGCACGCTCAAGGT-3′
F4:
(SEQ ID NO: 8)
5′-CACCATGGACCTTGAGCGTG-3′
R4:
(SEQ ID NO: 9)
5′-CTCAGCCGCCTTCAGCTTGTT-3′
F5:
(SEQ ID NO: 10)
5′-CACCATGCAGATTAAGACACTTACCAACAAG-3′
R5:
(SEQ ID NO: 11)
5′-GTAGCCAGACAGTTCGCTGAAAGTCT-3′

### Expression and Purification of Recombinant Allergens.

All recombinant proteins were expressed in ONE-SHOT BL21 STAR (DE3) cells (Invitrogen, Grand Island, N.Y.) induced with 1 mM isopropylthio-β-galactoside for four hours. Cells were harvested by centrifugation and stored at −80° C. overnight. Next day, pellets were dissolved in native buffer (50 mM Na phosphate, 0.5 M NaCl, 1 mg/ml lysozyme, protease inhibitors, pH 8.0). Cells were sonicated using the Misonix system (Misonix, Inc., Farmingdale, N.Y.) and cell debris was removed by centrifugation at 5000 rpm for 15 minutes. Proteins were extracted and purified using Ni-NTA purification system (Invitrogen, Grand Island, N.Y.) according to the manufacturer's instructions using native or denaturing buffer conditions. Proteins were dialyzed against phosphate-buffered saline (PBS, pH 7.4) and concentrations were determined by densitometric analysis of SDS-PAGE gels stained with coomasie brilliant blue, using natural shrimp tropomyosin (Indoor Biotechnologies, Inc., Charlottesville, Va.) as standard.

### Circular Dichroism Spectroscopy

Proteins dialyzed against PBS were adjusted to concentration of 200 μg/ml. Circular Dichroism spectroscopy was performed with a spectropolarimeter (Model 420, Aviv Biomedical Inc., Lakewood, N.J.) with a constant nitrogen flushing at 20° C. Proteins were scanned with a spectral range of 185-255 nm, with a step width of 0.2 nm and band width of 1 nm.

### Immunoblotting and Western Analysis

Proteins were fractionated by SDS-PAGE under reducing conditions using 4-12% gradient precast gels (Invitrogen, Grand Island, N.Y.) and bands were detected by coomasie staining. For immunoblotting, Pen a1 and fragments separated by SDS-PAGE were transferred onto nitrocellulose membranes using the iBlot (Bio-Rad Laboratories, Inc., Hercules, Calif.), blocked with 3% BSA in TBST (0.1% tween in TBS) and incubated overnight with the pool of patient sera diluted in blocking buffer. Bound IgE was detected using anti-IgE HRP (Invitrogen, Grand Island, N.Y.). For detection and quantification of purified IgE, elution fractions mixed with sample buffer without DTT were run on SDS-PAGE, transferred onto nitrocellulose membrane, and blocked and detected with anti-IgE HRP as described above. In between incubations, membranes were washed three times with TBST (Tris-Buffered Saline and Tween 20) for five minutes each with gentle shaking at room temperature.

### Digestion of Pen a1

rPen a1 was digested in 1.5 μl water, 0.5 μl of 10×SGF and 1 μl of pepsin at 0.1 μg/ml, 0.3 μg/ml, 1 μg/ml, 3 μg/ml, or 10 μg/ml. Digestion was performed by incubating samples at 37° C. for 10 minutes and digested samples were analyzed by SDS-PAGE and western blotting.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

Sequence Listing Free Text
SEQ ID NO: 1
MDAIKKKMQA MKLEKDNAMD RADTLEQQNK EANNRAEKSE
EEVHNLQKRM QQLENDLDQV QESLLKANIQ LVEKDKALSN
AEGEVAALNR RIQLLEEDLE RSEERLNTAT TKLAEASQAA
DESERMRKVL ENRSLSDEER MDALENQLKE ARFLAEEADR
KYDEVARKLA MVEADLERAE ERAETGESKI VELEEELRVV
GNNLKSLEVS EEKANQREEA YKEQIKTLTN KLKAAEARAE
FAERSVQKLQ KEVDRLEDEL VNEKEKYKSI TDELDQTFSE
LSGY

1. A recombinant hypoallergen comprising at least one amino acid modification compared to a corresponding wildtype allergen, wherein the recombinant hypoallergen:
binds to IgE that specifically binds to the allergen; and
induces release of histamine from basophils to a degree less than the wildtype allergen.

2. The recombinant hypoallergen of claim 1 wherein the allergen comprises Pen 1 a of Farfantepenaeus aztecus.

3. The recombinant hypoallergen of claim 2 wherein the hypoallergen is a fragment of Pen 1 a that comprises amino acids 1-79 of SEQ ID NO: 1.

4. The recombinant hypoallergen of claim 2 wherein the hypoallergen is a fragment of Pen 1 a that comprises amino acids 68-127 of SEQ ID NO:1.

5. The recombinant hypoallergen of claim 2 wherein the hypoallergen is a fragment of Pen 1 a that comprises amino acids 121-181 of SEQ ID NO:1.

6. The recombinant hypoallergen of claim 2 wherein the hypoallergen is a fragment of Pen 1 a that comprises amino acids 172-236 of SEQ ID NO:1.

7. The recombinant hypoallergen of claim 2 wherein the hypoallergen is a fragment of Pen 1 a that comprises amino acids 224-284 of SEQ ID NO:1.

8. The recombinant hypoallergen of claim 1 wherein the allergen comprises or is derived from Ara h 2 from Arachi hypogaea.

9. The recombinant hypoallergen of claim 1 wherein the allergen comprises or is derived from Jun a 1 from Juniperus ashei.

10. The recombinant hypoallergen of claim 1 wherein the allergen comprises or is derived from Fel d 1 of Felis domesticus.

11. The recombinant hypoallergen of claim 1 wherein the allergen comprises or is derived from Par j 1 of Parietaria judaica.

12. A pharmaceutical composition comprising:
a recombinant hypoallergen of claim 1; and
a pharmaceutically acceptable carrier.

13. A method of treating allergy in a subject to an allergen, the method comprising:
administering to the subject an amount of a recombinant hypoallergen in an amount effective to ameliorate at least one symptom or clinical sign of allergy to the allergen,
wherein the hypoallergen comprises at least one amino acid modification compared to a corresponding wildtype allergen, and wherein the recombinant hypoallergen:
binds to IgE that specifically binds to the allergen; and
induces release of histamine from basophils to a degree less than the wildtype allergen.

14. The use of a recombinant hypoallergen in the manufacture of a pharmaceutical composition for the treatment of allergy to a corresponding wildtype allergen.
